(19) United States
(12) Patent Application Publication
(10) Pub. No.: US 2023/0210995 A1
(43) Pub. Date: Jul. 6, 2023

(54) LOCALIZED EXPRESSION OF THERAPEUTIC NUCLEIC ACIDS IN LUNG EPITHELIAL CELLS

(71) Applicants: Cheung ANTHONY, Montreal (CA); Lora JOSE, Montreal (CA); ENGENE, INC., Montreal (CA)

(72) Inventors: Anthony CHEUNG, Montreal (CA); Jose Lora, Boston, MA (US)

(21) Appl. No.: 17/794,577

(22) PCT Filed: Jan. 22, 2021
(86) PCT No.: PCT/US2021/014763

§ 371 Date: Jul. 21, 2022

Related U.S. Application Data
(60) Provisional application No. 62/964,588, filed on Jan. 22, 2020.
(60) Provisional application No. 63/079,399, filed on Sep. 16, 2020.

(51) Int. Cl. A61K 47/36; A61K 48/00; C12N 15/63; A61K 9/00; A61K 9/12; A61K 9/51

(52) U.S. Cl. CPC A61K 47/36; A61K 48/0041; C12N 15/63; A61K 9/0043; A61K 9/12; A61K 9/5161

(57) ABSTRACT

Provided herein are methods and compositions for the treatment of lung disorders comprising the expression of therapeutic nucleic acid(s) in human airway epithelial cells, including the treatment of cystic fibrosis and disorders caused by expression of a mutated CFTR gene comprising the expression of functional CFTR in human airway epithelial cells.

### CROSS-REFERENCES TO RELATED APPLICATIONS

This application claims the benefit of priority to U.S. Provisional Application Nos. 62/964,588, filed Jan. 22, 2020, and 63/079,399, filed Sep. 16, 2020, the contents of which are hereby incorporated by reference in their entirety and for all purposes.

### FIELD OF THE INVENTION

The present disclosure relates to compositions and related methods for the localized expression of therapeutic nucleic acids encoding proteins of interest in airway epithelial cells, including functional CFTR protein for use in the treatment of individuals with cystic fibrosis or a disorder characterized by expression of unfunctional CFTR protein, and functional hAAT protein for the treatment of COPD and/or emphysema caused by AAT deficiency.

### BACKGROUND OF THE INVENTION

### Cystic Fibrosis

Cystic fibrosis is a progressive inherited disease caused by mutations of the cystic fibrosis transmembrane conductance regulator (CFTR). Individuals with cystic fibrosis have inherited mutated copies of the CFTR gene from both parents. The disease is characterized by the buildup of thick sticky mucus that can damage many of the body's organs. This can cause progressive damage to the respiratory system, clogging airways, causing coughing, wheezing, and inflammation. Over time, mucus buildup and infections, both bacterial and viral, result in permanent lung damage, including the formation of scar tissue (fibrosis) and cysts in the lungs. Cystic fibrosis patients also suffer chronic digestive problems. Some affected babies have meconium ileus, a blockage of the intestine that occurs shortly after birth. Other digestive problems result from a buildup of thick, sticky mucus in the pancreas, impairing its ability to produce insulin and digestive enzymes. Problems with digestion can lead to diarrhea, malnutrition, poor growth, and weight loss. In adolescence or adulthood, a shortage of insulin can cause a form of diabetes known as cystic fibrosis-related diabetes mellitus (CFRDM). Most men with cystic fibrosis have congenital bilateral absence of the vas deferens (CBAVD), a condition in which the tubes that carry sperm (the vas deferens) are blocked by mucus and do not develop properly. Men with CBAVD are unable to father children unless they undergo fertility treatment. Women with cystic fibrosis may experience complications in pregnancy as well.

### CFTR

The CFTR gene encodes a protein comprising two transmembrane domains, two nucleotide binding domains, and a unique regulatory domain resembling that of adenine nucleotide-bind cassette (ABC) transporters. CFTR functions as an anion channel, controlling ion and water secretion and absorption in epithelial tissues. CFTR channels are expressed at the apical membrane of epithelial cells lining the surface and submucosal glands of the conducting airways, and the lumens of the exocrine pancreas, gastrointestinal tract, sweat glands and some other epithelial and non-epithelial tissues. CFTR channels conduct Cl⁻ and HCO₃⁻ upon activation by protein kinase A (PKA_-dependent phosphorylation and play a central role in transepithelial ion/fluid transport and the regulation of the volume, salt content and pH of the airway surface liquid and other epithelial secretions (Mall et al., AJRCCM Articles in Press. Published 20-December 2019, 10.1164/rccm.201910-1943 SO). The CFTR mRNA and protein sequences are depicted FIG. 1 (Accession No. NM_000492.4) and FIG. 2 (NP_000483.3).

Over 2000 different mutations in the CFTR gene have been identified, of which more than 400 have been confirmed to cause cystic fibrosis. These mutations have been grouped into six classes.

Class 1A keeps the mRNA from being synthesized. These are generally the result of mutations in the DNA promoter, leading to the RNA polymerase not being able to bind to the DNA and therefore not copy the message into mRNA. As a result no CFTR protein is expressed. Examples of mutations that lead to no CFTR mRNA include the Dele2,3(21 kb) and 1717-1G→A.

With Class 1B mutations, the CFTR mRNA is produced but is damaged and cannot be used to express protein. These are largely the result of premature termination codons. This type of mutation results in the production of a shortened version of the CFTR protein, which is then degraded by the cell. Gly542X and Trp1282X are types of class 1B mutations.

Class 2 mutations are a result of misfolded proteins, which fail to reach the cell membrane and are degraded. Examples of class 2 mutations include Phe508del (also referred to as F508del), Asn1303Lys, and Ala561Glu. To correct the misfolded proteins and help them reach the cell membrane, treatments called CFTR correctors can be used. Some examples of CFTR correctors include lumacaftor/ivacaftor (marketed as Orkambi®), tezacaftor/ivacaftor (marketed as Symdeko®), and elexacaftor/tezacaftor/ivacaftor (Trikafta™) produced by Vertex Pharmaceuticals.

Class 3 mutations are another type of mutation that result in the production of a CFTR protein that makes it to the cell membrane but does not open correctly. This is often referred to as a “gating defect.” Gly551Asp, Ser549Arg, and Glyl349Asp are examples of mutations causing gating defects. Treatments called CFTR potentiators, such as ivacaftor (Kalydeco®), can be used to open the channels and/or keep them open for longer. Kalydeco is currently authorized by the EMA for the treatment of patients aged ≥2 years with G551D, the main gating mutation, accounting for ˜4-5% of the CF patient population, and an additional eight other gating mutations (G1244E, G1349D, G178R, G551S, S1251N, S1255P, S549N and S549R). Kalydeco is also indicated for the treatment of adult CF patients who have an R117H mutation in the CFTR gene.

Class 4 mutations result in a CFTR protein that makes it to the cell membrane and reacts to cell signaling to open, but the protein is misshapen and only allows a small amount of chloride ions to pass through. This reduction in chloride ion movement is called decreased conductance. Examples of such mutations include Arg117His, Arg334Trp, Arg 347Pro, and Ala455Glu. CFTR potentiators can also be helpful for these mutations to keep the channels open for longer to allow more chloride ions to flow through.

Class 5 mutations are alternative splicing mutations that do not change the conformation of the protein but alter its abundance by introducing promoter or splicing abnormalities. The incorrect versions that are expressed never make it to the cell surface, which leads to a reduction in the number of CFTR protein channels at the cell membrane. Class 5 mutations include 3272-26A→G, 3849+10 kg C→T. Possible treatments for this type of mutation include CFTR correctors to correct the misshapen CFTR proteins, CFTR potentiators to try and keep the working CFTR proteins open for longer, CFTR amplifiers to increase the amount of mRNA and therefore more CFTR protein being produced, or antisense oligonucleotides, which can have a number of different uses.

Class 6 mutations destabilize the channel in post-ER compartments and/or at the plasma membrane (PM), by reducing its conformational stability and/or generating additional internalization signals. Working CFTR protein is produced, but the protein configuration is not stable and will degrade too quickly once on the cell surface. This results in accelerated plasma membrane turnover and reduced apical plasma membrane expression. Class 6 mutations include 4326delTC, 4279insA, c.120de1123, and rPhe580del. Stabilizers are a class of treatment for this type of mutation. They work to inhibit enzymes that break down CFTR. A treatment called cavosonstat was being investigated for this use but failed to meet primary objectives in a Phase 2 clinical trial.

Cystic fibrosis therapies further include transmembrane conductance regulator (CFTR) modulator therapies designed to correct the malfunctioning protein made by the CFTR gene. Because different mutations cause different defects in the protein, the medications that have been developed so far are effective only in people with specific mutations. There are four CFTR modulators for people with certain CFTR mutations: ivacaftor (Kalydeco®)(Vertex), lumacaftor/ivacaftor (Orkambi®)(Vertex), tezacaftor/ivacaftor (Symdeko®)(Vertex), and elexacaftor/tezacaftor/ivacaftor (Trikafta™)(Vertex). Patents covering these drugs include, but are not limited to U.S. Pat. Nos. 7,495,103, 7,645,789, 7,776,905, U.S. 797303, U.S. Pat. Nos. 8,324,242, 8,354,427, 8,410,274, 8,415,387, 8,507,534, 8,598,181, 8,623,905, 8,629,162, 8,653,103, 8,716,338, 8,741,933, 8,754,224, 8,846,718, 8,883,206, 8,993,600, 9,012,496, 9,150,552, 9,192,606, 9,216,969, 9,670,163, 9,931,334, 9,974,781 and 10,022,352, 10,058,546, 10,081,621, 10,206,877, 10,239,867, and 10,272,046.

Ivacaftor (VX-770) is a CFTR potentiator, as such it helps ions flow through the cell membrane channel and improve the flow of water and salts across the cell membrane. It is used for the treatment of cystic fibrosis in people having one of several specific mutations in the cystic fibrosis transmembrane conductance regulator (CFTR) protein: E56K, G178R, S549R, K1060T, G1244E, P67L, E193K, G551D, A1067T, S1251N, R74W, L206W, G551S, G1069R, S1255P, D110E, R347H, D579G, R1070Q, D1270N, D110H, R352Q, S945L, R1070W, G1349D, R117C, A455E, S977F, F1074L, R117H, S549N, F1052V, D1152H. It has been reported that ivacaftor's specificity only benefits 5% of CF patients.

Lumacaftor, Tezacaftor, and Elexacaftor each work to restore function of the CFTR gene caused by the F508del mutation, which results in a defective misfolded CFTR protein. The F508del mutation results in a misfolded protein that is degraded by the cell before it can reach the cell membrane. Even if some of the defective protein reaches the cell membrane, it is unable to open correctly to allow the passage of chloride ions. F508del is the most common CFTR mutation, with nearly 90% of CF patients have at least one copy of the F508del mutation.

Lumacaftor (VX-809) acts as a chaperone during protein folding and increases the number of CFTR proteins that are trafficked to the cell surface. Lumacaftor works by increasing the stability of defective CFTR proteins, thereby helping them reach the cell membrane and stay there longer. But because it does not address the problem with the opening of the channel, lumacaftor is generally used in combination with ivacaftor, which acts on the defective proteins, helping them to open more often so that the salt-water balance across the cell surface is restored.

Tezacaftor (VX-661) helps move the cystic fibrosis transmembrane conductance regulator (CFTR) protein to the correct position on the cell surface. Tezacaftor is a corrector-type CFTR modulator. Its function is to correct the positioning of the CFTR protein on the cell surface to permit proper channel formation and improved flow of water and salts across the cell membrane.

Elexacaftor (VX-445) is a next-generation corrector, designed to treat the most common form of CF, those characterized by the F508del mutation. It is used in combination with tezacaftor and ivacaftor.

Existing supportive therapies for cystic fibrosis therapies include, but are not limited to oral or I.V. antibiotics including penicillins including amoxicillin and clavulanic acid (Augmentin®), dicloxacillin, Nafcillin and oxacillin, piperacillin/tazobactam (Zosyn®), cephalosporins including cephalexin, cefdinir, cefutoxime, ceftazidime, ceftazidime/avibactam, cefepime, cefoxitin, ceftolozane-tazobactam, ceftaroline, and cefazolin, carbapenems including meropenem, imipenem/cilastin, doripenem, meropenem-avibactam, and ertapenem, sulfa drugs including sulfamethoxazole and trimethoprim (Bactrim®), tetracyclines including tetracycline, doxycycline, minocycline, and tigecycline, vancomycin, clindamycin, linezolid, aminoglycosides including tobramycin, amikacin, and gentimicin, macrolides including azithromycin, quinolones including ciprofloxacin, moxifloxacin and levofloxacin; and others including aztreonam, Colistin®, rifampin, clindamycin. Mupirocin, clofazimine, and ethambutol

Other therapies include hydrators of the airway surface, mucolytics including hypertonic saline and dornase alfa (Pulmozyme®), inhaled antimicrobials, systemic anti-inflammatory treatments and nutritional support. In some cases lung transplants are performed. Gene therapies for cystic fibrosis are largely based on the delivery of a functional version of the CFTR gene to a cystic fibrosis patient via viral vectors, including lentiviral, adenoviral or adenoviral-associated vectors-based or liposomes.

Although therapies have extended the lives of cystic fibrosis patients, there remains a need for new compositions and methods providing reduced toxicity and improved efficacy, and in particular for treating those patients having mutations that are not addressed by the current therapies, e.g. Class I mutations. Ideally, these could be used in conjunction with current therapies to enhance therapy in the airway epithelial tissue.

### COPD

Chronic obstructive pulmonary disease (COPD) is an over-arching term for a number of lung disorders such as bronchiectasis, chronic bronchitis, and emphysema causing obstructed airflow from the lungs. Symptoms include breathing difficulty, cough, mucus (sputum) production and wheezing. As one example, the genetic mutation of human Alpha-1 Anti-Trypsin (hAAT) is the most commonly inherited form of emphysema seen in young people. It predisposes affected individuals to early emphysema and cirrhosis of the liver. To date, there are no definitive treatments, nor are there any efficient clinical management strategies. Existing and prospective therapies, have yet to demonstrate their efficacy to slow, halt or reverse the disease.

### SUMMARY OF THE INVENTION

The present invention resolves the still unmet need in the art for the effective localized expression of therapeutic proteins of interest in the lung epithelial tissues of a patient in need thereof, including, e.g., CFTR protein in cystic fibrosis patients and hAAT in COPD/emphysema patients, employing compositions comprising derivatized chitosan-nucleic acid polyplexes reversibly coated with a polyanion-containing block co-polymer for robust transfection of airway epithelial cells in the lung. In some embodiments, aerosolized pharmaceutical compositions comprising the subject derivatized chitosan-nucleic acid polyplexes reversibly coated with the polyanion-containing block co-polymer can be advantageously employed in the treatment methods provided herein.

In one aspect, the invention provides a composition comprising a derivatized chitosan-nucleic acid polyplex comprising amino-functionalized chitosan and a therapeutic nucleic acid construct encoding a protein of interest for treatment of a lung disorder, wherein said derivatized chitosan-nucleic acid polyplex further comprises a reversible coating comprising one or more polyanion-containing block co-polymers having at least one polyanionic anchor region and at least one hydrophilic tail region. In one embodiment, the nucleic acid encodes for CFTR protein, or a fragment thereof. In another embodiment, the nucleic acid encodes for hAAT protein, or a fragment thereof.

In preferred embodiments, the polyanion-containing block co-polymer is a linear diblock and/or triblock co-polymer. In preferred embodiments, the amino-functionalized chitosan further comprises a hydrophilic polyol. In some embodiments, the amino-functionalized chitosan comprises arginine. In some embodiments, the hydrophilic polyol is glucose or gluconic acid.

In some embodiments, the nucleic acid encoding a CFTR protein of interest comprises SEQ ID NO: 1 or NM_000492.4.3, or a fragment thereof. In some embodiments, the CFTR protein comprises SEQ ID NO:2 or NP_000483.3.

In some embodiments, the composition is used for localized expression of functional CFTR in the lung epithelial tissues of a patient in need thereof, comprising administering to said patient a therapeutically effective amount of a pharmaceutical composition comprising the compositions described herein. In some embodiments, the method is used to treat cystic fibrosis. In some embodiments, the compositions and methods described herein are used for the treatment of CF which is resistant to or not adequately treated by existing therapies. In an exemplary embodiment, the subject compositions and methods find advantageous use in the treatment of CF in patients having a Class 1 mutation, for whom current therapies are largely ineffective.

In some embodiments, the nucleic acid encoding a hAAT protein of interest comprises SEQ ID NO: 3 or K01396.1, or a fragment thereof. In some embodiments, the hAAT protein comprises SEQ ID NO:4 or AAB59375.1.

In some embodiments, the composition is used for localized expression of functional hAAT in lung epithelial tissues in a patient in need thereof, comprising administering to said patient a therapeutically effective amount of a pharmaceutical composition comprising the compositions described herein. In some embodiments, the method is used to treat COPD, including emphysema. In some embodiments, the compositions and methods described herein are used for the treatment of COPD (e.g. emphysema) which is resistant to or not adequately treated by existing therapies. In an exemplary embodiment, the subject compositions and methods find advantageous use in the treatment of emphysema in patients having a hAAT mutation, for whom current therapies are largely ineffective.

In another aspect, pharmaceutical compositions are provided comprising the subject derivatized chitosan-nucleic acid polyplexes reversibly coated with polyanion-containing block co-polymers having at least one polyanionic anchor region and at least one hydrophilic tail region, and preferably wherein said pharmaceutical compositions are aerosolized. In particularly preferred embodiments, the aerosolized composition is delivered using a mesh nebulizer. In some embodiments the aerosolized compositions for delivery via said mesh nebulizer comprises pegylated DD-X-DNA polyplexes in a trehalose solution.

In some embodiments, the aerosolized composition comprises an expression vector within a plasmid selected from the group consisting of: gWIZ, pVAX, NTC8382 NTC8685, or NTC9385R.

In some embodiments, the expression vector comprises one or more of the following elements consisting of a CMV-IE based promoter or enhancer or a lung tissue specific promotor (e.g. SCGB1A1 promoter), a synthetic Beta-globin-based intron, HTLV-1R, kanamycin selection or sucrose-based selection element, and an origin of replication.

In another aspect, the invention provides liquid formulations comprising derivatized chitosan nucleic acid polyplexes comprising amino-functionalized chitosan and at least one therapeutic nucleic acid in a trehalose solution for delivery as an aerosol via inhalation, wherein said therapeutic nucleic acid is for the treatment of a lung disorder and said polyplexes further comprise a reversible coating comprising one or more polyanion containing block co-polymers. In some embodiments, the amino-functionalized chitosan further comprises a hydrophilic polyol, preferably glucose or gluconic acid. In some embodiments, the amino-functionalized chitosan comprises arginine. In some embodiments, each polyanion-containing block co-polymer comprises at least one polyanionic anchor region and at least one hydrophilic tail region. In some embodiments, each polyanion-containing block co-polymer is a diblock and/or triblock co-polymer, preferably a linear diblock and/or triblock co-polymer.

In some embodiments, the polyplexes have an amino to phosphorus (N:P) ratio of between 5:1 and 15:1, or a N:P ratio of 7:1 or 10:1. In some embodiments, the amino to anion (N:A) molar ratio is from about 1:3 to about 1.7, more about 1:5. In some embodiments, the N:P:A ratio is about 10:1:7; about 10:1:3; or about 10:1:5. In some embodiments, the polyplexes comprise a DNA concentration of 0.1-2 mg/mL DNA. In some embodiments, the trehalose concentration is between 4% and 6%, preferably about 5%. In some embodiments, the liquid formulation has a pH between 5.0 and 8.0. In some embodiments, the liquid formulation has an osmolality between 150 and 250 mOsm/kg. In some embodiments, the liquid formulation is free of unbound DNA. In some embodiments, the polyplexes comprise between 75-100% supercoiled DNA, or between 85-95% supercoiled DNA. In some embodiments, the liquid formulation has a polydispersity index (PDI) of less than 0.5, 0.4, 0.3 or 0.25.

In another aspect, the invention provides kits comprising a therapeutically effective amount of the subject liquid formulations, a nebulizer, and instructions for use. In preferred embodiments, the nebulizer is a mesh nebulizer.

### INCORPORATION BY REFERENCE

All publications, patents, and patent applications mentioned in this specification are herein incorporated by reference to the same extent as if each individual publication, patent, or patent application was specifically and individually indicated to be incorporated by reference. In particular, U.S. 62/645,588, U.S. 63/079,399, PCT/IB2020/000175 and PCT/IB2020/000178 are incorporated by reference in their entirety.

### DEFINITIONS

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, where indicated, the term “about” indicates the designated value ±one standard deviation of that value.

The term “combinations thereof” includes every possible combination of elements to which the term refers.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder. For example, in an exemplary embodiment, the phrase “treating cystic fibrosis” refers to adding, improving, or restoring CFTR expression and/or function.

As used herein, the term “therapeutically effective amount” or “effective amount” refers to an amount of the subject compositions that when administered to a subject is effective to treat a disease or disorder. For example, in an exemplary embodiment, the phrase “effective amount” is used interchangeably with “therapeutically effective amount” or “therapeutically effective dose” and the like, and means an amount of a therapeutic agent that is effective for treating cystic fibrosis. Effective amounts of the compositions provided herein may vary according to factors such as the disease state, age, sex, weight of the animal.

As used herein, the term “subject” or “individual” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has cystic fibrosis, an autoimmune disease or condition, and/or an infection that can be treated with a protein provided herein. In some embodiments, the subject is a human that is suspected to have cystic fibrosis, an autoimmune disease or condition, and/or an infection.

“Chitosan” is a partially or entirely deacetylated form of chitin, a polymer of N-acetylglucosamine. Chitosans with any degree of deacetylation greater than 50% are used in the present invention.

Chitosan may be derivatized by functionalizing free amino groups at the sites of deacetylation. The derivatized chitosans described herein have a number of properties which are advantageous for a nucleic acid delivery vehicle including: they effectively bind and complex the negatively charged nucleic acids, they can be formed into nanoparticles of a controllable size, they can be taken up by the cells and they can release the nucleic acids at the appropriate time within the cells. Chitosans with any degree of functionalization between 1% and 50%. (Percent functionalization is determined relative to the number of free amino moieties on the chitosan polymer prior-to or in the absence of functionalization.) The degrees of deacetylation and functionalization impart a specific charge density to the functionalized chitosan derivative.

A polyol according to the present invention may have a 3, 4, 5, 6, or 7 carbon backbone and may have at least 2 hydroxyl groups. Such polyols, or combinations thereof, may be useful for conjugation to a chitosan backbone, such as a chitosan that has been functionalized with a cationic moiety (e.g., a molecule comprising an amino group such as, lysine, ornithine, a molecule comprising a guanidinium group, arginine, or a combination thereof).

The term “C₂-C₆ alkylene” as used herein refers to a linear or branched divalent hydrocarbon radical optionally containing one or more carbon-carbon multiple bonds. For the avoidance of doubt, the term “C₂-C₆ alkylene” as used herein encompasses divalent radicals of alkanes, alkenes and alkynes.

As used herein, unless otherwise indicated, the term “peptide” and “polypeptide” are used interchangeably.

The term “polypeptide” is used in its broadest sense to refer to conventional polypeptides (i.e., short polypeptides containing L or D-amino acids), as well as peptide equivalents, peptide analogs and peptidomimetics that retain the desired functional activity. Peptide equivalents can differ from conventional peptides by the replacement of one or more amino acids with related organic acids, amino acids or the like, or the substitution or modification of side chains or functional groups.

Peptidomimetics may have one or more peptide linkages replaced by an alternative linkage, as is known in the art. Portions or all of the peptide backbone can also be replaced by conformationally constrained cyclic alkyl or aryl substituents to restrict mobility of the functional amino acid sidechains, as is known in the art.

The polypeptides of this invention may be produced by recognized methods, such as recombinant and synthetic methods that are well known in the art. Techniques for the synthesis of peptides are well known and include those described in Merrifield, J. Amer. Chem. Soc. 85:2149-2456 (1963), Atherton, et al., Solid Phase Peptide Synthesis: A Practical Approach, IRL Press (1989), and Merrifield, Science 232:341-347 (1986).

As used herein, “linear polypeptide” refers to a polypeptide that lacks branching groups covalently attached to its constituent amino acid side chains. As used herein, “branched polypeptide” refers to a polypeptide that comprises branching groups covalently attached to its constituent amino acid side chains.

The “final functionalization degree” of cation or polyol as used herein refers to the percentage of cation (e.g., amino) groups on the chitosan backbone functionalized with cation (e.g., amino) or polyol, respectively. Accordingly, “α:β ratio”, “final functionalization degree ratio” (e.g., Arg final functionalization degree: polyol final functionalization degree ratio) and the like may be used interchangeably with the term “molar ratio” or “number ratio.”

Dispersed systems consist of particulate matter, known as the dispersed phase, distributed throughout a continuous medium. A “dispersion” of chitosan nucleic acid polyplexes is a composition comprising hydrated chitosan nucleic acid polyplexes, wherein polyplexes are distributed throughout the medium.

As used herein, a “pre-concentrated” dispersion is one that has not undergone the concentrating process to form a concentrated dispersion.

As used herein, “substantially free” of polyplex precipitate means that the composition is essentially free from particles that can be observed on visual inspection.

As used herein, physiological pH refers to a pH between 6 to 8.

By “chitosan nucleic acid polyplex” or its grammatical equivalents is meant a complex comprising a plurality of chitosan molecules and a plurality of nucleic acid molecules. In a preferred embodiment, the (e.g., dually-) derivatized-chitosan is complexed with said nucleic acid.

The term “polyethylene glycol” (“PEG”) as used herein is intended to mean a polymer of ethylene oxide having repeat units of —(CH2CH2-0)— and the general formula of HO— (CH2CH2-O)n—H.

The term “monomethoxy polyethylene glycol” (“mPEG”) as used herein is intended to mean a polymer of ethylene oxide having repeat units of —(CH2CH2-0)— and the general formula of CH3O—(CH2CH2-O)n—H, for example, a PEG capped at one end with a methoxy group.

The term “PEG-polyanion” (“PEG-PA”) as used herein refers to a polyanion such as PEG-polyglutamic acid, PEG-Hyaluronic acid, or PEG-polyaspartic acid.

### BRIEF DESCRIPTION OF THE DRAWINGS

The present disclosure is disclosed with reference to the accompanying drawings, wherein:

FIG. 1 illustrates the CFTR nucleic acid sequence.

FIG. 2 illustrates the CFTR protein sequence.

FIG. 3 illustrates the levels of GFP fluorescence seen in human bronchial cells transfected with a RXG 28:10 polyplex comprising a GFP expression vectors.

FIGS. 4A and 4B compare levels of luciferase expression seen in vivo in mice administered unpegylated (FIG. 4A) and pegylated (FIG. 4B) polyplex comprising a plasmid expressing Luc2.

FIGS. 5A and 5B illustrate the levels and duration of Luc2 expression by measurement of RLU intensity (FIG. 5A) and mRNA copy number (FIG. 5B).

FIG. 6 illustrates the dose response as measured in luciferase activity in mice administered a single dose of pegylated DDX comprising a luciferase reporter gene.

FIG. 7 compares levels of luciferase expression seen in vivo in mice administered naive, pegylated and unpegylated polyplexes.

FIG. 8 depicts the content recovery levels of DD-X polymer, PEG-PLE, and DNA content in polyplex formulations after nebulization.

FIG. 9 illustrates luciferase activity levels in mouse lungs 24 hours post-delivery of dispersed polyplexes comprising plasmid DNA packaged with either DDX or RXG polyplexes at concentrations from 0.125 mg/mL-1 mg/mL.

FIG. 10 illustrates luciferase activity in HBE cells following transfection with RXG.

FIGS. 11A and 11B show luciferase expression using an unoptimized vector. FIG. 11A shows durability of the response over 4 days. FIG. 11B shows the level of nanoparticle escape to peripheral tissues.

FIGS. 12A and 12B illustrate the dose-dependent increase in nanoparticle delivery (DNA copies in lung) (FIG. 12A) and mRNA expression (mRNA copies in lung) (FIG. 12B) following intratracheal administration in mice.

FIGS. 13A and 13B show DNA (FIG. 13A) and mRNA (FIG. 13B) copy number in mouse lung over 7 days post administration of nanoparticles.

FIG. 14 illustrates levels of dose-responsive mRNA expression of RXG-containing hCFTR or soCFTR in mouse lung.

FIGS. 15A-15C show results of FISH studies of mouse lungs 48 hours-post administration of nanoparticles.

FIG. 16A provides diagrams for jet and mesh nebulizers.

FIG. 16B provides product specifications for Vibronic and PDAP nebulizers.

FIG. 17 illustrates the % product recovery post-nebulization.

FIG. 18 illustrates the efficacy of nebulized formulations showing luciferase activity 24 hours after intranasal delivery in mice.

FIG. 19 shows the nebulized particle size for different compositions using Vibronic and PDAP nebulizers.

FIG. 20A provides the coding sequence for hAAT.

FIG. 20B provides the peptide sequence for hAAT.

FIG. 21 Illustrates hCFTR functionality following transfection in Hek293 cells.

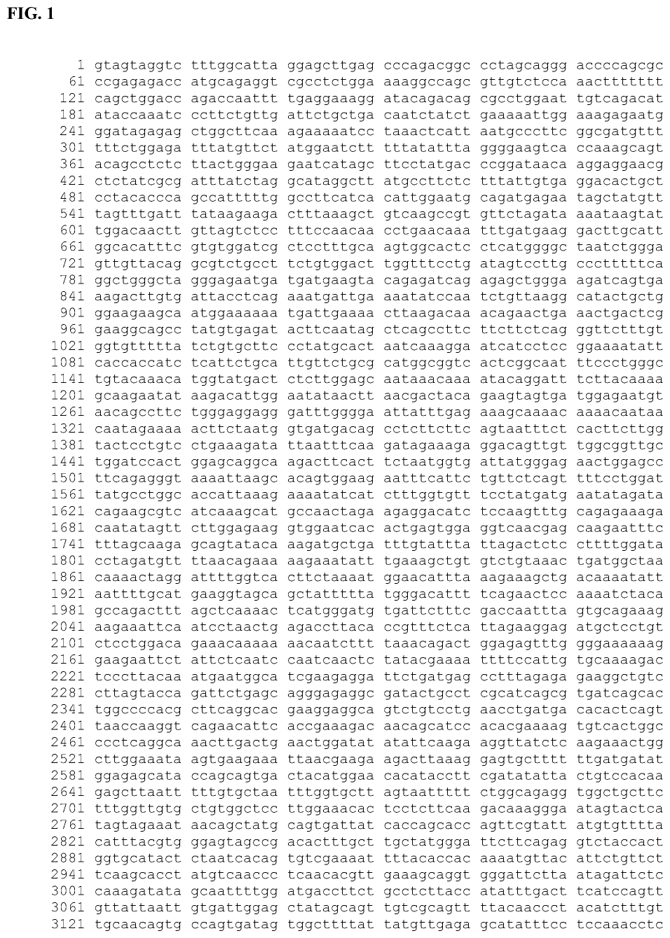

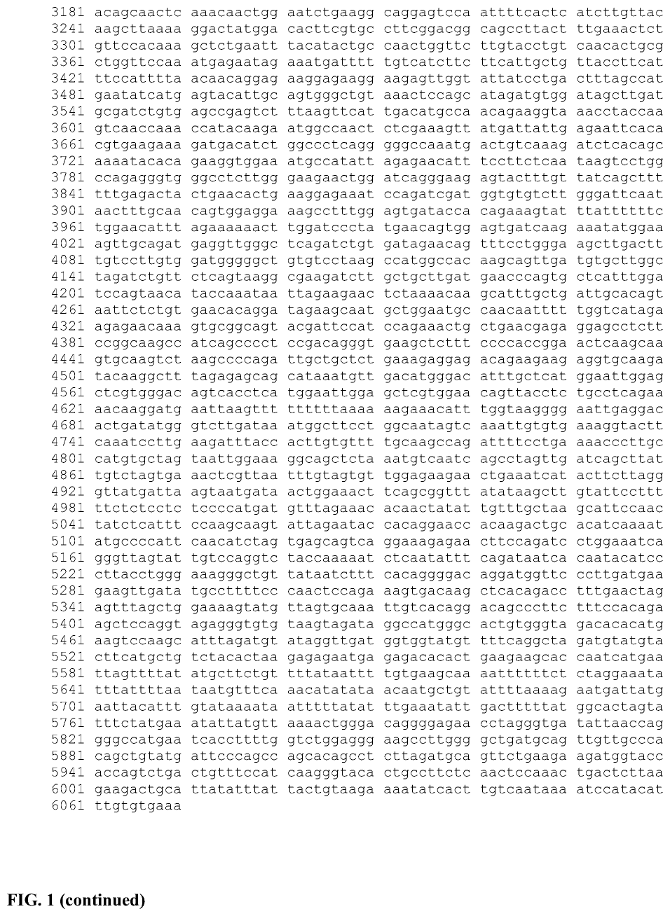

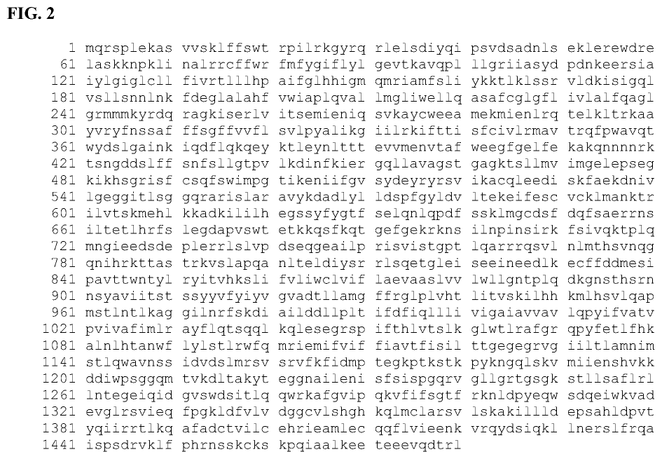

### DETAILED DESCRIPTION

The present invention contemplates localized expression of functional proteins of interest in airway epithelial tissues. Parenteral routes of protein therapies to airway epithelial tissues suffer from the lack of site specificity and systemic toxicity. Localized gene therapies at epithelial tissue present an attractive approach to promote local expression of therapeutic proteins while offering a less invasive treatments for high patient compliance. Unfortunately, however, clinical use of viral delivery vectors is limited by viral immunogenicity which diminishes efficacy after repeated dosing; the complexity and cost of vector production; logistical complications associated with clinical implementation (e.g. biosafety containment and cold-chain storage), and most importantly, the lack of stability and/or functionality when aerosolizing conventional nucleic acid delivery vehicles. The present invention provides a safe and effective non-viral vector platform for the transfection of mucosal tissues, e.g., the airways and lungs, that overcomes all of these limitations.

### Compositions

Provided herein are chitosan compositions comprising a chitosan-derivative nucleic acid nanoparticle (polyplex) in complex with a polyanion-containing block co-polymer, e.g. a diblock and/or triblock co-polymer coating, wherein individual polymer molecules comprise a negatively charged anchor region and one or more non-charged hydrophilic tail regions. Exemplary polymer molecules useful in the methods and compositions of the present invention are “PEG-PA” polymer molecules comprising a polyethylene glycol (PEG) portion and a polyanion (PA) portion.

1.1. Derivatized Chitosan

The chitosan component of the chitosan-derivative nucleic acid nanoparticle can be functionalized with a cationic functional group and/or a hydrophilic moiety. Chitosan functionalized with two different functional groups is referred to as dually derivitized chitosan (DD-chitosan). Exemplary DD-chitosans are functionalized with both a hydrophilic moiety (e.g., a polyol) and a cationic functional group (e.g., an amino group). Exemplary chitosan derivatives are also described in, e.g., U.S. 2007/0281904; and U.S. 2016/0235863, which are each incorporated herein by reference.

In one embodiment, the dually derivatized chitosan described herein comprises chitosan having a degree of deacetylation of at least 50%. In one embodiment, the degree of deacetylation is at least 60%, more preferably at least 70%, more preferably at least 80%, more preferably at least 90%, and most preferably at least 95%. In a preferred embodiment, the dually derivatized chitosan described herein comprises chitosan having a degree of deacetylation of at least 98%.

The chitosan derivatives described herein have a range of average molecular weights that are soluble at neutral and physiological pH, and include for the purposes of this invention molecular weights ranging from 3 — 110 kDa. Embodiments described herein feature lower average molecular weight of derivatized chitosans (<25 kDa, e.g., from about 5 kDa to about 25 kDa), which can have desirable delivery and transfection properties, and are small in size and have favorable solubility. A lower average molecular weight derivatized chitosan is generally more soluble than one with a higher molecular weight, the former thus producing a nucleic acid/chitosan complex that will release more easily the nucleic acid and provide increased transfection of cells. Much literature has been devoted to the optimization of all of these parameters for chitosan based delivery systems.

An ordinarily skilled artisan will recognize that chitosan refers to a plurality of molecules having a structure of Formula I, wherein n is any integer, and each R1 is independently selected from acetyl or hydrogen, wherein the degree of R1 selected from hydrogen is between 50% to 100%. Also, chitosan referred to as having an average molecular weight, e.g., of 3 kD to 110 kD, generally refers to a plurality of chitosan molecules having a weight average molecular weight of, e.g., 3 kD to 110 kD, respectively, wherein each of the chitosan molecules may have different chain lengths (n+2). It is also well recognized that chitosan referred to as “n-mer chitosan,” does not necessarily comprise chitosan molecules of Formula I, wherein each chitosan molecule has a chain length of n+2. Rather, “n-mer chitosan” as used herein refers a plurality of chitosan molecules, each of which may have different chain lengths, wherein the plurality has an average molecule weight substantially similar to or equal to a chitosan molecule having a chain length of n. For example, 24-mer chitosan may comprise a plurality of chitosan molecules, each having different chain lengths ranging from, e.g. 7-50, but which has a weight average molecular weight substantially similar or equivalent to a chitosan molecule having a chain length of 24.

A dually derivatized chitosan of the invention may also be functionalized with a polyol, or a hydrophilic functional group such as a polyol. Without wishing to be bound by theory, it is hypothesized that functionalization with a hydrophilic group such as a polyol which may help to increase the hydrophilicity of chitosan (including Arg-chitosan) and/or may donate a hydroxyl group. In some embodiments, the hydrophilic functional group of the chitosan-derivative nanoparticles is or comprises gluconic acid. See, e.g., WO 2013/138930. In some embodiments, the hydrophilic functional group of the chitosan-derivative nanoparticles is or comprises glucose. Additionally or alternatively, the hydrophilic functional group can comprise a polyol. See, e.g., U.S. 2016/0235863. Exemplary polyols for functionalization of chitosan are further described below.

The functionalized chitosan derivatives described herein include dually derivatized-chitosan compounds, e.g., cation-chitosan-polyol compounds. In general, the cation-chitosan-polyol compounds are functionalized with an amino-containing moiety, such as an arginine, lysine, ornithine, or molecule comprising a guanidinium, or a combination thereof. In certain embodiments, the cation-chitosan-polyol compounds have the following structure of Formula I:

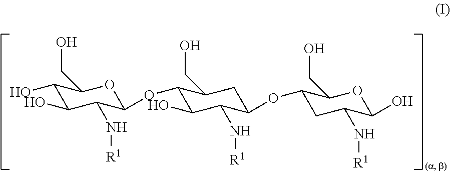

wherein n is an integer of 1 to 650,
α is the final functionalization degree of the cation moiety (e.g., a molecule comprising an amino group such as, lysine, ornithine, a molecule comprising a guanidinium group, arginine, or a combination thereof),
β is the final functionalization degree of polyol; and
each R¹ is independently selected from hydrogen, acetyl, a cation (e.g., arginine), and a polyol.

Preferably, a dually derivatized chitosan of the invention may be functionalized with the cationic amino acid, arginine.

In one embodiment, the chitosan-derivative nanoparticle comprises chitosan coupled with gluconic acid at a final functionalization degree of 1%, 2%, 4%, 7%, 8%, 10%, 15%, 20%, 25%, 30%, or greater. In one embodiment, the chitosan-derivative nanoparticle comprises chitosan coupled with glucose at a final functionalization degree of 1%, 2%, 4%, 7%, 8%, 10%, 15%, 20%, 25%, 30%, or greater. In one embodiment, the chitosan derivative nanoparticle comprises chitosan coupled with a cationic moiety (e.g., arginine) at a final functionalization degree of from about 1% to about 25%. In one embodiment, the chitosan derivative nanoparticle comprises chitosan coupled with a cationic moiety (e.g., arginine) at a final functionalization degree of from about 10% to about 40%.

In one embodiment, the chitosan derivative nanoparticle comprises chitosan coupled with a cationic moiety (e.g., arginine) at a final functionalization degree of from about 10% to about 35%. In one embodiment, the chitosan derivative nanoparticle comprises chitosan coupled with a cationic moiety (e.g., arginine) at a final functionalization degree of from about 20% to about 35%. In one embodiment, the chitosan derivative nanoparticle comprises chitosan coupled with a cationic moiety (e.g., arginine) at a final functionalization degree of from about 25% to about 35%. In one embodiment, the chitosan derivative nanoparticle comprises chitosan coupled with a cationic moiety (e.g., arginine) at a final functionalization degree of from about 25% to about 30%.

In one embodiment, the chitosan derivative nanoparticle comprises chitosan coupled with a cationic moiety (e.g., arginine) at a final functionalization degree of from about 15% to about 40%. In one embodiment, the chitosan derivative nanoparticle comprises chitosan coupled with a cationic moiety (e.g., arginine) at a final functionalization degree of from about 15% to about 35%. In one embodiment, the chitosan derivative nanoparticle comprises chitosan coupled with a cationic moiety (e.g., arginine) at a final functionalization degree of from about 15% to about 30%. In one embodiment, the chitosan derivative nanoparticle comprises chitosan coupled with a cationic moiety (e.g., arginine) at a final functionalization degree of from about 15% to about 28%.

In one embodiment, the chitosan derivative nanoparticle comprises chitosan coupled with a cationic moiety (e.g., arginine) at a final functionalization degree of from about 10% to about 35%. In one embodiment, the chitosan derivative nanoparticle comprises chitosan coupled with a cationic moiety (e.g., arginine) at a final functionalization degree of from about 10% to about 30%. In one embodiment, the chitosan derivative nanoparticle comprises chitosan coupled with a cationic moiety (e.g., arginine) at a final functionalization degree of from about 10% to about 28%. In one embodiment, the chitosan derivative nanoparticle comprises chitosan coupled with a cationic moiety (e.g., arginine) at a final functionalization degree of about 28%.

In one embodiment, the chitosan-derivative nanoparticle comprises chitosan coupled with gluconic acid at a final functionalization degree of from about 2% to about 30%, from about 5% to about 30%, from about 7.5% to about 30%, from about 5% to about 25%, from about 5% to about 22%, from about 5% to about 20%, from about 5% to about 15%, or from about 5% to about 10%. In one embodiment, the chitosan-derivative nanoparticle comprises chitosan coupled with gluconic acid at a final functionalization degree of from about 7.5% to about 25%, from about 7.5% to about 20%, from about 7.5% to about 15%, or from about 7.5% to about 12%. In one embodiment, the chitosan-derivative nanoparticle comprises chitosan coupled with gluconic acid at a final functionalization degree of about 10%.

In one embodiment, the chitosan-derivative nanoparticle comprises chitosan coupled with hydrophilic polyol at a final functionalization degree of from about 2% to about 30%, from about 5% to about 30%, from about 7.5% to about 30%, from about 5% to about 25%, from about 5% to about 22%, from about 5% to about 20%, from about 5% to about 15%, or from about 5% to about 10%. In one embodiment, the chitosan-derivative nanoparticle comprises chitosan coupled with hydrophilic polyol at a final functionalization degree of from about 7.5% to about 25%, from about 7.5% to about 20%, from about 7.5% to about 15%, or from about 7.5% to about 12%. In one embodiment, the chitosan-derivative nanoparticle comprises chitosan coupled with hydrophilic polyol at a final functionalization degree of about 10%.

In one embodiment, the chitosan-derivative nanoparticle comprises chitosan coupled with glucose at a final functionalization degree of from about 2% to about 30%, from about 5% to about 30%, from about 7.5% to about 30%, from about 5% to about 25%, from about 5% to about 22%, from about 5% to about 20%, from about 5% to about 15%, or from about 5% to about 10%. In one embodiment, the chitosan-derivative nanoparticle comprises chitosan coupled with glucose at a final functionalization degree of from about 7.5% to about 25%, from about 7.5% to about 20%, from about 7.5% to about 15%, or from about 7.5% to about 12%. In one embodiment, the chitosan-derivative nanoparticle comprises chitosan coupled with glucose at a final functionalization degree of about 10%.

In one embodiment, the chitosan-derivative nanoparticle comprises chitosan coupled with cation (e.g., arginine) at a final functionalization degree of from about 2% to about 40% and hydrophilic polyol (e.g., glucose or gluconic acid) at a final functional degree of from about 2% to about 30%. In one embodiment, the chitosan-derivative nanoparticle comprises chitosan coupled with cation (e.g., arginine) at a final functionalization degree of from about 5% to about 40% and hydrophilic polyol (e.g., glucose or gluconic acid) at a final functional degree of from about 5% to about 25%. In one embodiment, the chitosan-derivative nanoparticle comprises chitosan coupled with cation (e.g., arginine) at a final functionalization degree of from about 7.5% to about 40% and hydrophilic polyol (e.g., glucose or gluconic acid) at a final functional degree of from about 7.5% to about 20%. In one embodiment, the chitosan-derivative nanoparticle comprises chitosan coupled with cation (e.g., arginine) at a final functionalization degree of from about 10% to about 40% and hydrophilic polyol (e.g., glucose or gluconic acid) at a final functional degree of from about 7.5% to about 15%, or about 10%.

In one embodiment, the chitosan-derivative nanoparticle comprises chitosan coupled with cation (e.g., arginine) at a final functionalization degree of from about 2% to about 35% and hydrophilic polyol (e.g., glucose or gluconic acid) at a final functional degree of from about 2% to about 30%. In one embodiment, the chitosan-derivative nanoparticle comprises chitosan coupled with cation (e.g., arginine) at a final functionalization degree of from about 5% to about 35% and hydrophilic polyol (e.g., glucose or gluconic acid) at a final functional degree of from about 5% to about 25%. In one embodiment, the chitosan-derivative nanoparticle comprises chitosan coupled with cation (e.g., arginine) at a final functionalization degree of from about 7.5% to about 35% and hydrophilic polyol (e.g., glucose or gluconic acid) at a final functional degree of from about 7.5% to about 20%. In one embodiment, the chitosan-derivative nanoparticle comprises chitosan coupled with cation (e.g., arginine) at a final functionalization degree of from about 10% to about 35% and hydrophilic polyol (e.g., glucose or gluconic acid) at a final functional degree of from about 7.5% to about 15%, or about 10%.

In one embodiment, the chitosan-derivative nanoparticle comprises chitosan coupled with cation (e.g., arginine) at a final functionalization degree of from about 10% to about 30% and hydrophilic polyol (e.g., glucose or gluconic acid) at a final functional degree of from about 2% to about 30%. In one embodiment, the chitosan-derivative nanoparticle comprises chitosan coupled with cation (e.g., arginine) at a final functionalization degree of from about 12% to about 30% and hydrophilic polyol (e.g., glucose or gluconic acid) at a final functional degree of from about 5% to about 25%. In one embodiment, the chitosan-derivative nanoparticle comprises chitosan coupled with cation (e.g., arginine) at a final functionalization degree of from about 14% to about 30% and hydrophilic polyol (e.g., glucose or gluconic acid) at a final functional degree of from about 7.5% to about 20%. In one embodiment, the chitosan-derivative nanoparticle comprises chitosan coupled with cation (e.g., arginine) at a final functionalization degree of from about 15% to about 30% and hydrophilic polyol (e.g., glucose or gluconic acid) at a final functional degree of from about 7.5% to about 15%, or about 10%.

In one embodiment, the chitosan-derivative nanoparticle comprises chitosan coupled with cation (e.g., arginine) at a final functionalization degree of about 25% and hydrophilic polyol (e.g., glucose or gluconic acid) at a final functional degree of from about 7.5% to about 15%. In one embodiment, the chitosan-derivative nanoparticle comprises chitosan coupled with cation (e.g., arginine) at a final functionalization degree of about 28% and hydrophilic polyol (e.g., glucose or gluconic acid) at a final functional degree of from about 7.5% to about 15%. In one embodiment, the chitosan-derivative nanoparticle comprises chitosan coupled with cation (e.g., arginine) at a final functionalization degree of about 25% and hydrophilic polyol (e.g., glucose or gluconic acid) at a final functional degree of from about 5% to about 20%. In one embodiment, the chitosan-derivative nanoparticle comprises chitosan coupled with cation (e.g., arginine) at a final functionalization degree of about 28% and hydrophilic polyol (e.g., glucose or gluconic acid) at a final functional degree of from about 5% to about 20%.

In a preferred embodiment, the chitosan-derivative nanoparticle comprises chitosan coupled with cation (e.g., arginine) at a final functionalization degree of about 14% and hydrophilic polyol (e.g., glucose or gluconic acid) at a final functional degree of about 10%. In a preferred embodiment, the chitosan-derivative nanoparticle comprises chitosan coupled with cation (e.g., arginine) at a final functionalization degree of about 15% and hydrophilic polyol (e.g., glucose or gluconic acid) at a final functional degree of about 12%. In another preferred embodiment, the chitosan-derivative nanoparticle comprises chitosan coupled with arginine at a final functionalization degree of about 14% and glucose at a final functional degree of about 10%. In another preferred embodiment, the chitosan-derivative nanoparticle comprises chitosan coupled with arginine at a final functionalization degree of about 15% and glucose at a final functional degree of about 12%.

In a preferred embodiment, the chitosan-derivative nanoparticle comprises chitosan coupled with cation (e.g., arginine) at a final functionalization degree of about 28% and hydrophilic polyol (e.g., glucose or gluconic acid) at a final functional degree of about 10%. In another preferred embodiment, the chitosan-derivative nanoparticle comprises chitosan coupled with arginine at a final functionalization degree of about 28% and glucose at a final functional degree of about 10%.

In some embodiments, where appropriate, DD-chitosan includes DD-chitosan derivatives, e.g., DD chitosan that incorporate an additional functionalization, e.g., DD-chitosan with an attached ligand. “Derivatives” will be understood to include the broad category of chitosan-based polymers comprising covalently modified N-acetyl-D-glucosamine and/or D-glucosamine units, as well as chitosan-based polymers incorporating other units, or attached to other moieties. Derivatives are frequently based on a modification of the hydroxyl group or the amine group of glucosamine, such as done with arginine-functionalized chitosan. Examples of chitosan derivatives include, but are not limited to, trimethylated chitosan, thiolated chitosan, galactosylated chitosan, alkylated chitosan, PEI-incorporated chitosan, uronic acid modified chitosan, glycol chitosan, and the like. For further teaching on chitosan derivatives, see, e.g., pp.63-74 of “Non-viral Gene Therapy”, K. Taira, K. Kataoka, T. Niidome (editors), Springer-Verlag Tokyo, 2005, ISBN 4-431-25122-7; Zhu et al., Chinese Science Bulletin, December 2007, vol. 52 (23), pp. 3207-3215; and Varma et al., Carbohydrate Polymers 55 (2004) 77-93.

1.2. Derivatized Chitosan Nucleic Acid Polyplex

The chitosan-derivative nanoparticle compositions generally contain at least one nucleic acid molecule, and preferably a plurality of such nucleic acid molecules. Typical nucleic acid molecules comprise phosphorous as a component of the nucleic acid backbone, e.g., in the form of a plurality of phosphodiesters or derivatives thereof (e.g., phosphorothioate). The proportion of cation-functionalized chitosan-derivative to nucleic acid can be characterized by a cation (+) to phosphorous (P) molar ratio, wherein the (+) refers to the cation of the cation-functionalized chitosan-derivative and the (P) refers to the phosphorous of the nucleic acid backbone. Typically, the (+):(P) molar ratio is selected such that the chitosan-derivative-nucleic acid complex has a positive charge in the absence of the polyanion-containing block co-polymer reversible coating. Thus, the (+):(P) molar ratio is generally greater than 1. In preferred embodiments, the (+):(P) molar ratio is greater than 1.5, at least 2, or greater than 2. In certain preferred embodiments, the (+):(P) molar ratio is greater than 2.

In some cases, the (+):(P) molar ratio is, or is about, 3:1. In some cases, the (+):(P) molar ratio is, or is about, 4:1. In some cases, the (+):(P) molar ratio is, or is about, 5:1. In some cases, the (+):(P) molar ratio is, or is about, 6:1. In some cases, the (+):(P) molar ratio is, or is about, 7:1. In some cases, the (+):(P) molar ratio is, or is about, 8:1. In some cases, the (+):(P) molar ratio is, or is about, 9:1. In some cases, the (+):(P) molar ratio is, or is about, 10:1.

In some cases, the (+):(P) molar ratio is from greater than 1 to no more than about 20:1, from about 2 to no more than about 20:1, or from about 2 to no more than about 10:1. In some cases, the (+):(P) molar ratio is from greater than about 2 to no more than about 20:1, or from greater than about 2 to no more than about 10:1. In some cases, the (+):(P) molar ratio is from about 3 to no more than about 20:1, from about 3 to no more than about 10:1, from about 3 to no more than about 8:1, or from about 3 to no more than about 7:1. In some cases, the (+):(P) molar ratio is from about 3 to no more than 20:1, from about 3 to no more than 10:1, from about 3 to no more than 8:1, or from about 3 to no more than 7:1.

In certain embodiments, the (+):(P) molar ratio is 100:1, preferably less than 100:1. For example, in certain embodiments, (+):(P) molar ratio can be from greater than 1 to less than or equal to 100:1. In some cases, the (+):(P) molar ratio can be from greater than 2 to less than or equal to 100:1. In some cases, the (+):(P) molar ratio can be from greater than or equal to 3 to less than or equal to 100:1. In some cases, the (+):(P) molar ratio can be from greater than or equal to 5 to less than or equal to 100:1. In some cases, the (+):(P) molar ratio can be from greater than or equal to 7 to less than or equal to 100:1. In some cases, the (+):(P) molar ratio can be from greater than 2 to less than or equal to 50:1. In some cases, the (+):(P) molar ratio can be from greater than or equal to 3 to less than or equal to 50:1. In some cases, the (+):(P) molar ratio can be from greater than or equal to 5 to less than or equal to 50:1. In some cases, the (+):(P) molar ratio can be from greater than or equal to 7 to less than or equal to 50:1. In some cases, the (+):(P) molar ratio can be from greater than 2 to less than or equal to 25:1. In some cases, the (+):(P) molar ratio can be from greater than or equal to 3 to less than or equal to 25:1. In some cases, the (+):(P) molar ratio can be from greater than or equal to 5 to less than or equal to 25:1. In some cases, the (+):(P) molar ratio can be from greater than or equal to 7 to less than or equal to 25:1.

In some embodiments, the cationic functional group of the chitosan-derivative nanoparticles is or comprises an amino group. Examples of such amino-functionalized chitosan-derivative nanoparticles include, but are not limited to, those containing chitosan that is functionalized with: a guanidinium or a molecule comprising a guanidinium group, a lysine, an ornithine, an arginine, or a combination thereof. In preferred embodiments, the cationic functional group is an arginine. The proportion of amino-functionalized chitosan-derivative to nucleic acid can be characterized by an amino (N) to phosphorous (P) molar ratio, wherein the (N) refers to the nitrogen atom of the amino group in the amino-functionalized chitosan-derivative and the (P) refers to the phosphorous of the nucleic acid backbone. Typically, the N:P molar ratio is selected such that the chitosan-derivative-nucleic acid complex, in the absence of PEG-PA polymer molecules, has a positive charge at a physiologically relevant pH. Thus, the N:P molar ratio is generally greater than 1. In preferred embodiments, the N:P molar ratio is greater than 1.5, at least 2, or greater than 2. In certain preferred embodiments, the N:P molar ration is greater than 2.

In some cases, the N:P molar ratio is, or is about, 3:1. In some cases, the N:P molar ratio is, or is about, 4:1. In some cases, the N:P molar ratio is, or is about, 5:1. In some cases, the N:P molar ratio is, or is about, 6:1. In some cases, the N:P molar ratio is, or is about, 7:1. In some cases, the N:P molar ratio is, or is about, 8:1. In some cases, the N:P molar ratio is, or is about, 9:1. In some cases, the N:P molar ratio is, or is about, 10:1.

In some cases, the N:P molar ratio is from greater than 1 to no more than about 20:1, from about 2 to no more than about 20:1, or from about 2 to no more than about 10:1. In some cases, the N:P molar ratio is from greater than about 2 to no more than about 20:1, or from greater than about 2 to no more than about 10:1. In some cases, the N:P molar ratio is from about 3 to no more than about 20:1, from about 3 to no more than about 10:1, from about 3 to no more than about 8:1, or from about 3 to no more than about 7:1. In some cases, the N:P molar ratio is from about 3 to no more than 20:1, from about 3 to no more than 10:1, from about 3 to no more than 8:1, or from about 3 to no more than 7:1.

In certain embodiments, the N:P molar ratio is 100:1, preferably less than 100:1. For example, in certain embodiments, N:P molar ratio can be from greater than 1 to less than or equal to 100:1. In some cases, the N:P molar ratio can be from greater than 2 to less than or equal to 100:1. In some cases, the N:P molar ratio can be from greater than or equal to 3 to less than or equal to 100:1. In some cases, the N:P molar ratio can be from greater than or equal to 5 to less than or equal to 100:1. In some cases, the N:P molar ratio can be from greater than or equal to 7 to less than or equal to 100:1. In some cases, the N:P molar ratio can be from greater than 2 to less than or equal to 50:1. In some cases, the N:P molar ratio can be from greater than or equal to 3 to less than or equal to 50:1. In some cases, the N:P molar ratio can be from greater than or equal to 5 to less than or equal to 50:1. In some cases, the N:P molar ratio can be from greater than or equal to 7 to less than or equal to 50:1. In some cases, the N:P molar ratio can be from greater than 2 to less than or equal to 25:1. In some cases, the N:P molar ratio can be from greater than or equal to 3 to less than or equal to 25:1. In some cases, the N:P molar ratio can be from greater than or equal to 5 to less than or equal to 25:1. In some cases, the N:P molar ratio can be from greater than or equal to 7 to less than or equal to 25:1.

In a preferred embodiment, the subject polyplexes have amine to phosphate (N/P) ratio of 2 to 100, e.g., 2 to 50, e.g., 2 to 40, e.g., 2 to 30, e.g., 2 to 20, e.g., 2 to 5. Preferably, the N/P ratio is inversely proportional to the molecular weight of the chitosan, i.e., a smaller molecular weight (e.g., dually) derivatized-chitosan requires a higher N/P ratio, and vice versa.

A nucleic acid of the present invention will generally contain phosphodiester bonds, although in some cases nucleic acid analogs are included that may have alternate backbones or other modifications or moieties incorporated for any of a variety of purposes, e.g., stability and protection. Other analog nucleic acids contemplated include those with non-ribose backbones. In addition, mixtures of naturally occurring nucleic acids, analogs, and both can be made. The nucleic acids may be single stranded or double stranded or contain portions of both double stranded or single stranded sequence. Nucleic acids include but are not limited to DNA, RNA and hybrids where the nucleic acid contains any combination of deoxyribo- and ribo-nucleotides, and any combination of bases, including uracil, adenine, thymine, cytosine, guanine, inosine, xanthanine, hypoxanthanine, isocytosine, isoguanine, etc. Nucleic acids include DNA in any form, RNA in any form, including triplex, duplex or single-stranded, anti-sense, siRNA, ribozymes, deoxyribozymes, polynucleotides, oligonucleotides, chimeras, microRNA, and derivatives thereof. Nucleic acids include artificial nucleic acids, including but not limited to, peptide nucleic acid (PNA), phosphorodiamidate morpholino oligo (PMO), locked nucleic acid (LNA), glycol nucleic acid (GNA) and threose nucleic acid (TNA). It will be appreciated that, for artificial nucleic acids that do not comprise phosphorous, an equivalent measure of the (+):P or N:P ratio can be approximated by the number of nucleotide (or nucleotide analog) bases.

In a preferred embodiment, the polyplexes of the compositions comprise chitosan molecules having an average molecular weight of less than 110 kDa, more preferably less than 65 kDa, more preferably less than 50 kDa, more preferably less than 40 kDa, and most preferably less than 30 kDa before functionalization. In some embodiments, polyplexes of the compositions comprise chitosan having an average molecular weight of less than 15 kDa, less than 10 kDa, less than 7 kDa, or less than 5 kDa before functionalization.

In a preferred embodiment, the polyplexes comprise chitosan molecules having on average less than 680 glucosamine monomer units, more preferably less than 400 glucosamine monomer units, more preferably less than 310 glucosamine monomer units, more preferably less than 250 glucosamine monomer units, and most preferably less than 190 glucosamine monomer units. In some embodiments, the polyplexes comprise chitosan molecules having on average less than 95 glucosamine monomer units, less than 65 glucosamine monomer units, less than 45 glucosamine monomer units, or less than 35 glucosamine monomer units.

Chitosan, and (e.g., dually) derivatized-chitosan nucleic acid polyplexes may be prepared by any method known in the art, including but not limited to those described herein.

1.2.1. Nucleic Acids

As described above, the chitosan polyplexes can contain a plurality of nucleic acids. In one embodiment, the nucleic acid component comprises a therapeutic nucleic acid. The subject (e.g., dually) derivatized-chitosan nucleic acid polyplexes are amenable to the use of any therapeutic nucleic acid known in the art. Therapeutic nucleic acids include therapeutic RNAs, which are RNA molecules capable of exerting a therapeutic effect in a mammalian cell. Therapeutic RNAs include, but are not limited to, messenger RNAs, antisense RNAs, siRNAs, short hairpin RNAs, micro RNAs, and enzymatic RNAs. Therapeutic nucleic acids include, but are not limited to, nucleic acids intended to form triplex molecules, protein binding nucleic acids, ribozymes, deoxyribozymes, and small nucleotide molecules.

Many types of therapeutic RNAs are known in the art. For example, see Meng et al., A new developing class of gene delivery: messenger RNA-based therapeutics, Biomater. Sci.,5, 2381-2392, 2017; Grimm et al., Therapeutic application of RNAi is mRNA targeting finally ready for prime time? J. Clin. Invest., 117:3633-3641, 2007; Aagaard et al., RNAi therapeutics: Principles, prospects and challenges, Adv. Drug Deliv. Rev., 59:75-86, 2007; Dorsett et al., siRNAs: Applications in functional genomics and potential as therapeutics, Nat. Rev. Drug Discov., 3:318-329, 2004. These include double-stranded short interfering RNA (siRNA).

In some embodiments, the therapeutic nucleic acids include therapeutic DNAs which are DNA molecules capable of exerting a therapeutic effect in a mammalian cell. Therapeutic nucleic acids include, but are not limited to, nucleic acids intended to form triplex molecules, protein binding nucleic acids, ribozymes, deoxyribozymes, and small nucleotide molecules.

Therapeutic nucleic acids also include nucleic acids encoding therapeutic proteins, including cytotoxic proteins and prodrugs.

In a preferred embodiment, the nucleic acid component comprises a therapeutic nucleic acid construct. The therapeutic nucleic acid construct is a nucleic acid construct capable of exerting a therapeutic effect. Therapeutic nucleic acid constructs may comprise nucleic acids encoding therapeutic proteins, as well as nucleic acids that produce transcripts that are therapeutic RNAs. A therapeutic nucleic acid may be used to effect genetic therapy by serving as a replacement or enhancement for a defective gene or to compensate for lack of a particular gene product, by encoding a therapeutic product. A therapeutic nucleic acid may also inhibit expression of an endogenous gene. A therapeutic nucleic acid may encode all or a portion of a translation product, and may function by recombining with DNA already present in a cell, thereby replacing a defective portion of a gene. It may also encode a portion of a protein and exert its effect by virtue of co-suppression of a gene product. In some embodiments, the therapeutic nucleic acid is selected from those disclosed in U.S. 2011/0171314, which is expressly incorporated herein by reference.

In some embodiments, the therapeutic nucleic acid encodes a therapeutic protein that is selected from the group consisting of hormones, enzymes, cytokines, chemokines, antibodies, mitogenic factors, growth factors, differentiation factors, factors influencing cell apoptosis, factors influencing inflammation, and factors influencing the immune response (immunomodulators). Preferred therapeutic nucleic acids encode proteins finding use in the treatment of lung disorders, e.g., cystic fibrosis, COPD, asthma and the like, including, e.g., CFTR or hAAT, either alone or in conjunction with additional therapeutic proteins.

1.2.1.1. Expression Control Regions

In a preferred embodiment, a polyplex of the invention comprises a therapeutic nucleic acid, which is a therapeutic construct, comprising an expression control region operably linked to a coding region. The therapeutic construct produces therapeutic nucleic acid, which may be therapeutic on its own, or may encode a therapeutic protein.

In some embodiments, the expression control region of a therapeutic construct possesses constitutive activity. In a number of preferred embodiments, the expression control region of a therapeutic construct does not have constitutive activity. This provides for the dynamic expression of a therapeutic nucleic acid. By “dynamic” expression is meant expression that changes over time. Dynamic expression may include several such periods of low or absent expression separated by periods of detectable expression. In a number of preferred embodiments, the therapeutic nucleic acid is operably linked to a regulatable promoter. This provides for the regulatable expression of therapeutic nucleic acids.

Expression control regions comprise regulatory polynucleotides (sometimes referred to herein as elements), such as promoters and enhancers, which influence expression of an operably linked therapeutic nucleic acid.

Expression control elements included herein can be from bacteria, yeast, plant, or animal (mammalian or non-mammalian). Expression control regions include full-length promoter sequences, such as native promoter and enhancer elements, as well as subsequences or polynucleotide variants that retain all or part of full-length or non-variant function (e.g., retain some amount of nutrient regulation or cell/tissue-specific expression). As used herein, the term “functional” and grammatical variants thereof, when used in reference to a nucleic acid sequence, subsequence or fragment, means that the sequence has one or more functions of native nucleic acid sequence (e.g., non-variant or unmodified sequence). As used herein, the term “variant” means a sequence substitution, deletion, or addition, or other modification (e.g., chemical derivatives such as modified forms resistant to nucleases).

As used herein, the term “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Some expression control regions confer regulatable expression to an operably linked therapeutic nucleic acid. A signal (sometimes referred to as a stimulus) can increase or decrease expression of a therapeutic nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

Numerous regulatable promoters are known in the art. Preferred inducible expression control regions include those comprising an inducible promoter that is stimulated with a small molecule chemical compound. In one embodiment, an expression control region is responsive to a chemical that is orally deliverable but not normally found in food. Particular examples can be found, for example, in U.S. Pat. Nos. 5,989,910; 5,935,934; 6,015,709; and 6,004,941.

Promoter/enhancer sequences of particular interest are described in Table 1, below.

TABLE 1

| Promoter/ | |
|---|---|
| enhancer | |
| sequence | Description |
| CMV | Cytomegalovirus immediate early enhancer and |
| | promoter |
| EF1α | Human elongation factor (EF)-1α promoter |
| CMV/EF1α | CMV enhancer + core EF1α promoter |
| 2 × CMV/EF1α | 2 × CMV enhancer + core EF1α promoter |
| CAG | CMV enhancer + promoter, first exon and first intron |
| | of chicken beta-actin gene + splice acceptor of the |
| | rabbit beta globin gene |
| CBA | Chicken beta-actin promoter |
| CBh | CMV enhancer + modified chicken beta actin + hybrid |
| | intron of CBA and minute virus of mice (MVM) |
| EF1α/HTLV | Human EF1α core promoter + R segment and part of |
| | U5 sequence (R’-U5) of human T-cell leukemia virus |
| | Type 1 Long Terminal Repeat |
| CMV/EF1α/ | CMV enhancer + Human elongation factor (EF)-1α |
| HTLV | promoter + R segment and part of U5 sequence (R’-U5) |
| | of human T-cell leukemia virus Type 1 Long Terminal |
| | Repeat |
| UbC | Human ubiquitin C promoter |
| UbB | Human ubiquitin B promoter |
| CMV/UbC | CMV enhancer + Human ubiquitin C promoter |
| CMV/UbB | CMV enhancer + Human ubiquitin B promoter |
| PGK | Phosphoglycerate promoter |

In one embodiment, the therapeutic construct further comprises an integration sequence. In one embodiment, the therapeutic construct comprises a single integration sequence. In another embodiment, the therapeutic construct comprises a first and a second integration sequence for integrating the therapeutic donor nucleic acid or a portion thereof into the genome of a target cell. In a preferred embodiment, the integration sequence(s) is functional in combination with a means for integration.

Suitable means for integration include, but are not limited to transposon/transposase systems, recombinase systems, meganuclease systems including CRISPR-based systems, and systems that utilize an integrating viral vector, component thereof, or combination thereof. Suitable transposon/transposase systems include systems that utilize a mariner or sleeping beauty element, or a variant thereof. See, U.S. Pat. No. 9,228,180; U.S. 2019/0169638; and U.S. 2016/0264949. Suitable recombinase systems include those that utilize a FLP, Cre, tC31, R, lambda, TP901-1, or R4 recombinase, or a variant thereof. See, U.S. 2005/0153392; and U.S. Pat. No. 8,586,361. Suitable meganuclease systems include but are not limited to those that utilize Cas9 or Cas12 or a variant thereof a gRNA, sgRNA, and/or tracrRNA; Argonaute or a variant thereof, a zinc finger nuclease or variant thereof; a TALEN or a variant thereof; or a homing endonuclease or variant thereof, or a combination thereof, See, U.S. 2019/0185831; U.S. 2019/0055549; U.S. 2018/0171359; WO 2020/003006; U.S. 2020/00010519; U.S. 2020/0017878; U.S. 2017/0022507; U.S. 2018/0179547; U.S. 2014/0234289; U.S. 20100086533; U.S. 2011/0064717; and U.S. 2016/0367588.

In the context of a methods employing integration, the therapeutic donor nucleic acid encoding CFTR or a functional fragment thereof can encode a functional fragment that replaces a null mutation or nonsense mutation of an endogenous CFTR locus. In the context of a methods employing integration, the therapeutic donor nucleic acid encoding CFTR or a functional fragment thereof can encode a functional fragment that replaces a class 1 (e.g., class 1A or class 1B) mutation of an endogenous CFTR locus.

In some embodiments, one or more integration components, including but not limited to a donor nucleic acid (e.g., therapeutic nucleic acid encoding CFTR or a functional fragment thereof) and/or a transposase, recombinase, or meganuclease can be delivered in the form of a polyplex formulation comprising chitosan and nucleic acid as described herein. In some embodiments, one or more integration components, including but not limited to a donor nucleic acid (e.g., therapeutic nucleic acid encoding CFTR or a functional fragment thereof) and/or a transposase, recombinase, or meganuclease can be delivered via a viral vector, e.g., in the form of a polyplex formulation comprising chitosan and viral vector nucleic acid as described herein. In some embodiments, donor nucleic acid optionally in combination or simultaneously or sequentially with a transposase, recombinase, or meganuclease is delivered with an integrating viral vector nucleic acid. Suitable integrating viral vector nucleic acids include, but are not limited to, an AAV vector, a retroviral vector (e.g., a gamma retroviral vector), or a lentiviral vector. See, U.S. 2018/0271783; U.S. 2015/0203870; U.S. 2020/0017878; and WO/2006/036465.

In an exemplary embodiment, a subject is administered at least one donor nucleic acid containing polyplex formulation, wherein the at least one nucleic acid comprises a therapeutic donor nucleic acid comprising an expression control region operably linked to a coding region nucleic acid encoding a CFTR gene or functional fragment thereof, and wherein the subject is simultaneously or sequentially administered a nucleic acid encoding a meganuclease, recombinase, or transposase operably linked to a promoter. In a preferred embodiment, the subject is administered a nucleic acid encoding a CRISPR/Cas meganuclease, such as a CRISPR/Cas9 or Cas12 meganuclease, and required components thereof, such as a guide nucleic acid (e.g., gRNA, tracrRNA, and/or sgRNA) operably linked to a promoter.

In some cases, the subject is administered a first polyplex formulation comprising a therapeutic donor nucleic acid encoding CFTR or a functional fragment thereof and a second polyplex formulation comprising a nucleic acid encoding a meganuclease, recombinase, or transposase (e.g., a CRISPR/Cas meganuclease and/or guide nucleic acid). In some embodiments, the therapeutic donor nucleic acid encoding CFTR and the nucleic acid encoding a meganuclease, recombinase, or transposase (e.g., a CRISPR/Cas meganuclease and/or guide nucleic acid) is administered simultaneously in a single polyplex formulation or as an admixture of two different polyplex formulations. In some embodiments, the therapeutic donor nucleic acid encoding CFTR and the nucleic acid encoding a meganuclease, recombinase, or transposase are the same nucleic acid. In some embodiments, the therapeutic donor nucleic acid encoding CFTR and the nucleic acid encoding a meganuclease, recombinase, or transposase are operably linked to a different promoter and in the same contiguous nucleic acid. In a preferred embodiment, the therapeutic donor nucleic acid encoding CFTR and the nucleic acid encoding a meganuclease are different nucleic acids, e.g., administered in the same or a different polyplex formulation.

In some cases, the subject is administered a first polyplex formulation comprising a therapeutic donor nucleic acid encoding hAAT or a functional fragment thereof and a second polyplex formulation comprising a nucleic acid encoding a meganuclease, recombinase, or transposase (e.g., a CRISPR/Cas meganuclease and/or guide nucleic acid). In some embodiments, the therapeutic donor nucleic acid encoding hAAT and the nucleic acid encoding a meganuclease, recombinase, or transposase (e.g., a CRISPR/Cas meganuclease and/or guide nucleic acid) is administered simultaneously in a single polyplex formulation or as an admixture of two different polyplex formulations. In some embodiments, the therapeutic donor nucleic acid encoding hAAT and the nucleic acid encoding a meganuclease, recombinase, or transposase are the same nucleic acid. In some embodiments, the therapeutic donor nucleic acid encoding CFTR and the nucleic acid encoding a meganuclease, recombinase, or transposase are operably linked to a different promoter and in the same contiguous nucleic acid. In a preferred embodiment, the therapeutic donor nucleic acid encoding CFTR and the nucleic acid encoding a meganuclease are different nucleic acids, e.g., administered in the same or a different polyplex formulation.

In some embodiments of the invention, the therapeutic construct is comprised within a plasmid comprising an origin, a multicloning site and a selectable marker. In some embodiments, plasmids of less than 10 kb are desirable. In some embodiments the plasmids used are suitable for gene therapy in human patients, and/or are engineered for high levels of transient gene expression in mammalian tissues. In preferred embodiments, the plasmid is selected from the group consisting of the Nanoplasmid™ (e.g. NTC9385 plasmid (Nature Technology), gWIZ plasmid (Genlantis), or pVAX1 plasmid (Thermofisher Scientific). See, e.g, U.S. Pat. Nos. 6,027,722, 6,287,863, 6,410,220, 6,573,091, 9,012,226, 9,017,966, 9,018,012, 9,109,012, 9,487,788, 9,487,789, 9,506,082, 9,550,998, 9,725,725, 9,737,620, 9,950,081, 10,047,365, 10,144,935, and 10,167,478. In some embodiments, the plasmid has been “retrofitted” to remove antibiotic selection agents and/or to increase expression levels.

In one embodiment, the subject composition further comprises a non-therapeutic construct in addition to a therapeutic construct, wherein the non-therapeutic construct comprises a nucleic acid sequence encoding a means for integration operably linked to a second expression control region. This second expression control region and the expression control region operably linked to the therapeutic nucleic acid may be the same or different. The encoded means for integration is preferably selected from the group consisting of mariner, sleeping beauty, FLP, Cre, ΦC31, R, lambda, and means for integration from integrating viruses such as AAV, retroviruses, and lentiviruses.

For further teaching, see WO 2008/020318, which is expressly incorporated herein in its entirety by reference. In one embodiment, the nucleic acid of the (e.g., dually) derivatized-chitosan nucleic acid polyplex is an artificial nucleic acid.

Preferred artificial nucleic acids include, but are not limited to, peptide nucleic acid (PNA), phosphorodiamidate morpholino oligo (PMO), locked nucleic acid (LNA), glycol nucleic acid (GNA) and threose nucleic acid (TNA).

In one embodiment, the nucleic acid of the DD-chitosan nucleic acid polyplex is a therapeutic nucleic acid. In one embodiment, the therapeutic nucleic acid is a therapeutic RNA. Preferred therapeutic RNAs include, but are not limited to, antisense RNA, siRNA, short hairpin RNA, micro RNA, and enzymatic RNA.

In one embodiment, the therapeutic nucleic acid is DNA.

In one embodiment, the therapeutic nucleic acid comprises a nucleic acid sequence encoding a therapeutic protein.

1.3 Polyols

Chitosan-derivative nanoparticles can be functionalized with a polyol. Polyols useful in the present invention in general are typically hydrophilic. In some cases, the chitosan-derivative nanoparticles are functionalized with a cationic component such as an amino group and with a polyol. Such chitosan-derivative nanoparticles functionalized with a cationic moiety such as an amino group and a polyol are referred to as “dually-derivatized chitosan nanoparticles.”

In some embodiments, the chitosan-derivative nanoparticle comprises a polyol of Formula II:

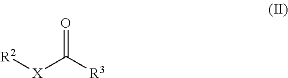

wherein:
R² is selected from: H and hydroxyl;
R³ is selected from: H and hydroxyl; and
X is selected from: C₂-C₆ alkylene optionally substituted with one or more hydroxyl substituents.

In some embodiments, the chitosan-derivative nanoparticle comprises a polyol of Formula II:

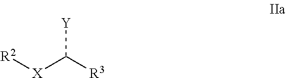

wherein:
—Y is ═O or —H₂
R² is selected from: H and hydroxyl;
R³ is selected from: H and hydroxyl; and
X is selected from: C₁-C₅ alkylene optionally substituted with one or more hydroxyl substituents and C₂-C₆ alkylene optionally substituted with one or more hydroxyl substituents.

In one embodiment, a polyol according to the present invention having 3 to 7 carbons may have one or more carbon-carbon multiple bonds. In a preferred embodiment, a polyol according to the present invention comprises a carboxyl group. In a further preferred embodiment, a polyol according to the present invention comprises an aldehyde group. A skilled artisan will recognize that when a polyol according to the present invention comprises an aldehyde group, such polyol encompasses both the open-chain conformation (aldehyde) and the cyclic conformation (hemiacetal).

Non-limiting examples of a polyols include gluconic acid, threonic acid, glucose and threose. Examples of other such polyols, which may have a carboxyl and/or aldehyde group, or may be a saccharide or acid form thereof, are described in more detail in U.S. Pat. No. 10,046,066, the disclosure of which is expressly incorporated by reference herein. A skilled artisan will recognize that the polyols are not limited to a specific stereochemistry.

In a preferred embodiment, the polyol may be selected from the group consisting of 2,3-dihydroxylpropanoic acid; 2,3,4,5, 6,7-hexahydroxylheptanal ; 2,3,4,5, 6-pentahydroxylhexanal ; 2,3,4,5-tetrahydroxylhexanal; and 2,3-dihydroxylpropanal.

In a preferred embodiment, the polyol may be selected from the group consisting of D-glyceric acid, L-glyceric acid, L-glycero-D-mannoheptose, D-glycero-L-mannoheptose, D-glucose, L-glucose, D-fucose, L-fucose, D-glyceraldehyde, and L-glyceraldehyde.

1.3.Polymer:Polyplex Compositions

Chitosan polyplexes can be mixed with a plurality of polymers, the polymers comprising a hydrophilic, non-charged portion, and a negatively charged (anionic) portion. As described above, the chitosan polyplexes are formulated to have a positive charge in the absence of, or prior to, complexing with the anionic portion-containing polymer. Thus under suitable conditions, the polymer component will form a reversible charge:charge complex with the chitosan-derivative nucleic acid polyplexes. In some embodiments, the polymers of the polymer component are unbranched. In some embodiments, the polymers are branched. In some cases, the polymer component comprises a mixture of branched and unbranched polymers.

In some embodiments, the polymer component is released from the chitosan polyplex after administration, after entering a cell, and/or after endocytosis. Without wishing to be bound by theory, it is hypothesized that the polyplex:polymer compositions thus formed by complexing polyplex and the anionic portion-containing polymer can provide improved in vitro, in solution, and/or in vivo stability without substantially interfering with transfection efficiency. In some embodiments, the polyplex:polymer compositions thus formed can provide reduced muco-adhesive properties as compared to, e.g., otherwise identical, polyplexes without the polymer component.

In a preferred embodiment, the polyplex:polymer compositions have a low net positive, neutral, or net negative zeta potential (from about +10 mV to about −20 mV) at physiological pH. Such compositions can exhibit reduced aggregation in physiological conditions and reduced non-specific binding to ubiquitous anionic components in vivo. Said properties can enhance migration of such composition (e.g., enhanced diffusion in mucus) to contact the cell and result in enhanced intracellular release of nucleic acid.

In a preferred embodiment, the polyplex:polymer particle compositions have an average hydrodynamic diameter of less than 1000 nm, more preferably less than 500 nm and most preferably less than 200 nm. In certain embodiments, the polyplex:polymer particle compositions have an average hydrodynamic diameter of from 50 nm to no more than 1000 nm, preferably from 50 nm to no more than 500 nm and most preferably from 50 nm to no more than 200 nm. In certain embodiments, the polyplex:polymer particle compositions have an average hydrodynamic diameter of from 50 nm to no more than 175 nm, preferably from 50 nm to no more than 150 nm. In certain embodiments, the polyplex:polymer particle compositions have an average hydrodynamic diameter of from 75 nm to no more than 1000 nm, preferably from 75 nm to no more than 500 nm and most preferably from 75 nm to no more than 200 nm. In certain embodiments, the polyplex:polymer particle compositions have an average hydrodynamic diameter of from 75 nm to no more than 175 nm, preferably from 75 nm to no more than 150 nm. In certain embodiments, the polyplex:polymer particle compositions have an average hydrodynamic diameter of greater than 100 nm and less than 175 nm.

In one embodiment, the polyplex:polymer compositions have a % supercoiled DNA content of 80%, at least 80%, or preferably 90%, more preferably at least 90%.

In one embodiment, the polyplex:polymer compositions have an average zeta potential of between +10 mV to -10 mV at a physiological pH, most preferably between +5 mV to −5 mV at a physiological pH.

The polyplex:polymer compositions are preferably homogeneous in respect of particle size. Accordingly, in a preferred embodiment, the composition has a low average polydispersity index (“PDI”). In an especially preferred embodiment, a dispersion of the polyplex:polymer composition has a PDI of less than 0.5, more preferably less than 0.4, more preferably less than 0.3, yet more preferably less than 0.25, and most preferably less than 0.2.

In some cases, a dispersion of the polyplex:polymer composition exhibits one or more of the foregoing PDI, average zeta potential, % supercoil DNA, or average particle size (nm) or size range after one or more freeze thaw cycles. In some cases, a dispersion of the polyplex:polymer composition exhibits one or more of the foregoing PDI, average zeta potential, % supercoil DNA, or average particle size (nm) or size range after storage in solution for at least 48 h at 4° C. In some cases, a dispersion of the polyplex:polymer composition exhibits one or more of the foregoing PDI, average zeta potential, % supercoil DNA, or average particle size (nm) or size range after storage in solution for at least for 2 weeks, or more at 4° C.

In some cases, a dispersion of the polyplex:polymer composition exhibits one or more of the foregoing PDI, average zeta potential, % supercoil DNA, or average particle size (nm) or size range after lyopholization and rehydration. In some cases, a dispersion of the polyplex:polymer composition exhibits one or more of the foregoing PDI, average zeta potential, % supercoil DNA, or average particle size (nm) or size range after spray drying and rehydration. In some cases, a dispersion of the polyplex:polymer composition exhibits one or more of the foregoing PDI, average zeta potential, % supercoil DNA, or average particle size (nm) or size range when concentrated (e.g., by ultrafiltration such as tangential flow filtration) to a nucleic acid concentration of at least 250 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 1,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 25,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 2,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 5,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 10,000 μg/mL.

In general, the polyplex:polymer compositions described herein, exhibit favorable solution behavior (e.g., stability and/or non-aggregation) as measured by PDI or mean particle size even in the absence of excipients such as lyoprotectants, cryoprotectants, surfactants, rehydration or wetting agents, and the like. In some cases, the polyplex:polymer compositions described herein exhibit favorable solution behavior (e.g., stability and/or non-aggregation) as measured by PDI or mean particle size in physiological fluids or simulated physiological fluids. For example, in some embodiments, the polyplex:polymer compositions described herein are stable in simulated intestinal fluid, in mammalian urine, and/or when stored in a mammalian bladder (e.g., and in contact with urine).

As described above, the polyplex:polymer compositions described herein are preferably substantially size stable in the composition. In a preferred embodiment, a composition of the invention comprises polyplex:polymer particles that increase in average diameter by less than 100%, more preferably less than 50%, and most preferably less than 25%, at room temperature for 6 hours, more preferably 12 hours, more preferably 24 hours, and most preferably 48 hours. In a particularly preferred embodiment, a composition of the invention comprises polyplex:polymer particles that increase in average diameter by less than 25% at room temperature for at least 24 hours or at least 48 hours.

The polyplex:polymer particles of the subject compositions are preferably substantially size stable under cooled conditions. In a preferred embodiment, a composition of the invention comprises polyplex:polymer particles that increase in average diameter by less than 100%, more preferably less than 50%, and most preferably less than 25%, at 2-8 degrees Celsius for 6 hours, more preferably 12 hours, more preferably 24 hours, and most preferably 48 hours.

The polyplex:polymer particles of the subject compositions are preferably substantially size stable under freeze-thaw conditions. In a preferred embodiment, a composition of the invention comprises polyplexes that increase in average diameter by less than 100%, more preferably less than 50%, and most preferably less than 25% at room temperature for 6 hours, more preferably 12 hours, more preferably 24 hours, and most preferably 48 hours following thaw from frozen at -20 to -80 degrees Celsius.

In a preferred embodiment, the composition has a nucleic acid concentration greater than 0.5 mg/ml, and is substantially free of precipitated polyplex. More preferably, the composition has a nucleic acid concentration of at least 0.6 mg/ml, more preferably at least 0.75 mg/ml, more preferably at least 1.0 mg/ml, more preferably at least 1.2 mg/ml, and most preferably at least 1.5 mg/ml, and is substantially free of precipitated polyplex. In another preferred embodiment, the composition has a nucleic acid concentration greater than 2 mg/ml, and is substantially free of precipitated polyplex. More preferably, the composition has a nucleic acid concentration of at least 2.5 mg/ml, more preferably at least 5 mg/ml, more preferably at least 10 mg/ml, more preferably at least 15 mg/ml, and most preferably about 25 mg/ml, and is substantially free of precipitated polyplex. In some embodiments, the composition has a nucleic acid concentration from 0.5 mg/mL to about 25 mg/mL, and is substantially free of precipitated polyplex. In some embodiments, the composition has a nucleic acid concentration of ≤ about 25 mg/mL, and is substantially free of precipitated polyplex. The compositions can be hydrated. In a preferred embodiment, the composition is substantially free of uncomplexed nucleic acid.

In a preferred embodiment, the polyplex:polymer particle composition is isotonic. Achieving isotonicity, while maintaining polyplex stability, is highly desirable in formulating pharmaceutical compositions, and these preferred compositions are well suited to pharmaceutical formulation and therapeutic applications.

In certain embodiments, the polyplex:polymer particle composition can be uncoated to release all or part of the, e.g., PEG, polymer coat by reducing pH. In certain embodiments, the polymer coat is released by incubating the particle under a pH condition that is below the pKa of the polyanionic anchor region of the polymer. For example, where the polymer coat is polyglutamate, the polymer coat can be released by incubating the particle at a pH below the pKa of polyglutamate, such as a pH of less than about 4.25. In certain embodiments, the polymer coat can be released by incubating the particle under a pH condition that is at least 0.25 pH units or at least 0.5 pH units below the pKa of the polyanion anchor region of the polymer coat.

In certain embodiments, the polyplex:polymer particle composition can be uncoated to release all or part of the, e.g., PEG, polymer coat by reducing pH. In certain embodiments, the polymer coat is released by incubating the particle under a pH condition that is below the pKa of the polyanionic anchor region of the polymer. For example, where the polymer coat is polyglutamate, the polymer coat can be released by incubating the particle at a pH below the pKa of polyglutamate, such as a pH of less than about 4.25. In certain embodiments, the polymer coat can be released by incubating the particle under a pH condition that is at least 0.25 pH units or at least 0.5 pH units below the pKa of the polyanion anchor region of the polymer coat.

In certain embodiments, the polyplex:polymer particle composition can be uncoated to release all or part of the, e.g., PEG, polymer coat by subjecting the particle to a high ionic strength.

Without wishing to be bound by theory, it is hypothesized that certain physiological conditions can promote partial, substantial (>50%), or total uncoating of reversibly PEGylated chitosan DNA polyplexes described herein. For example, low pH conditions in certain subcellular compartments (e.g., endosome, early endosome, late endosome, or lysosome) can facilitate release of the polymer coat. As another example, certain extracelluar conditions can promote partial, substantial (>50%), or total uncoating of reversibly PEGylated chitosan DNA polyplexes described herein. In some cases, the high ionic strength and/or acidic pH conditions typically encounted in certain positions in the alimentary canal can promote partial, substantial (>50%), or total uncoating of reversibly PEGylated chitosan DNA polyplexes described herein. It will be appreciated that anion charge density and/or pKa of the anionic anchor region of a polymer can be adjusted to promote or inhibit release under intended conditions. Optimized reversibly PEGylated particle compositions can be identified by assaying for stability and transfection efficiency using assays described herein.

The compositions comprising chitosan polyplex complexed with the anionic portion-containing polymer can be characterized by the ratio of cationic functional groups of the (e.g., dually) derivatized-chitosan polyplex (+) to anion moieties of the polymer (−), referred to as the “(+):(−) molar ratio”. This (+):(−) molar ratio can vary from greater than about 1:100 to less than about 10:1.

In certain embodiments, the (+):(−) molar ratio can be from greater than about 1:75 to less than about 8:1. In some cases, the (+):(−) molar ratio can be from greater than 1:10 to less than 10:1. In some cases, the (+):(−) molar ratio can be from, or from about, 1:10 to, or to about, 10:1. In some cases, the (+):(−) molar ratio can be from, or from about, 1:8 to, or to about, 8:1. In certain embodiments, the (+):(−) molar ratio can be from greater than 1:50 to less than about 10:1. In some cases, the (+):(−) molar ratio can be from greater than 1:25 to less than about 10:1. In some cases, the (+):(−) molar ratio can be from greater than 1:10 to less than about 7:1. In some cases, the (+):(−) molar ratio can be from greater than 1:8 to less than about 7:1. In some cases, the (+):(−) molar ratio can be from greater than 1:8 to less than about 6:1.

In certain embodiments, where the cationic functional group of the (e.g., dually) derivatized-chitosan polyplex is an amino moiety, the compositions comprising chitosan polyplex complexed with the anionic portion-containing polymer can be characterized by the ratio of amino groups of the (e.g., dually) derivatized-chitosan polyplex (N) to anion (A) moieties of the polymer, referred to as the “N:A molar ratio”. This N:A molar ratio can vary from greater than about 1:100 to less than about 10:1.

In certain embodiments, the N:A molar ratio can be from greater than about 1:75 to less than about 8:1. In some cases, the N:A molar ratio can be from greater than 1:10 to less than 10:1. In some cases, the N:A molar ratio can be from, or from about, 1:10 to, or to about, 10:1. In some cases, the N:A molar ratio can be from, or from about, 1:8 to, or to about, 8:1. In certain embodiments, the N:A molar ratio can be from greater than 1:50 to less than about 10:1. In some cases, the N:A molar ratio can be from greater than 1:25 to less than about 10:1. In some cases, the N:A molar ratio can be from greater than 1:10 to less than about 7:1. In some cases, the N:A molar ratio can be from greater than 1:8 to less than about 7:1. In some cases, the N:A molar ratio can be from greater than 1:8 to less than about 6:1.

Additionally or alternatively, the compositions comprising chitosan polyplex complexed with the anionic portion-containing polymer can be characterized by a three-component ratio of cationic functional groups of the (e.g., dually) derivatized-chitosan polyplex (+) to phosphours atoms of the nucleic acid (P) to anion moieties of the polymer (−), referred to as the “(+):P:(−) molar ratio”.

In certain embodiments, where (+):P is from at least 2:1 to no more than 20:1, the molar ratio of (+):(−) can vary from at least 1:40 to about 40:1. In certain embodiments, where (+):P is from at least 2:1 to no more than 20:1, the molar ratio of (+):(−) can vary from at least 1:40 to about 1:10. In some embodiments, where (+):P is from at least 2:1 to no more than 20:1, the molar ratio of (+):(−) can vary from at least 1:25 to about 25:1. In some embodiments, where (+):P is from at least 2:1 to no more than 20:1, the molar ratio of (+):(−) can vary from at least 1:25 to about 1:10. In some cases, where (+):P is from at least 2:1 to no more than 20:1, the molar ratio of (+):(−) can vary from at least 1:20 to about 20:1. In some cases, where (+):P is from at least 2:1 to no more than 20:1, the molar ratio of (+):(−) can vary from at least 1:20 to about 1:10. In some cases, where (+):P is from at least 2:1 to no more than 20:1, the molar ratio of (+):(−) can vary from at least 1:10 to about 10:1. In some cases, where (+):P is from at least 2:1 to no more than 20:1, the molar ratio of (+):(−) can vary from at least 1:25 to about 2:1. In some cases, where (+):P is from at least 2:1 to no more than 20:1, the molar ratio of (+):(−) can vary from at least 1:20 to about 1:1.

In certain preferred embodiments, (+):P:(−) is from 3:1:3.5 to 3:1:17.5. In certain preferred embodiments, (+):P:(−) is from 5:1:3.5 to 5:1:17.5. In certain preferred embodiments, (+):P:(−) is from 7:1:3.5 to 7:1:17.5. In certain preferred embodiments, (+):P:(−) is about 3:1:3.5, 3:1:7, 3:1:10, 3:1:15, 3:1:17.5, or 3:1:20. In certain preferred embodiments, (+):P:(−) is about 5:1:3.5, 5:1:7, 5:1:10, 5:1:15, 5:1:17.5, or 5:1:20. In certain preferred embodiments, (+):P:(−) is about 7:1:3.5, 7:1:7, 7:1:10, 7:1:15, 7:1:17.5, or 7:1:20. In certain preferred embodiments, (+):P:(−) is about 10:1:10, 10:1:15, 10:1:20, 10:1:25, 10:1:30, or 10:1:40.

One of skill in the art will appreciate that amino-functionalized chitosan polyplex particles in complex with the anionic portion-containing polymer can be characterized by a three-component ratio of amino functional groups of the (e.g., dually) derivatized-chitosan polyplex (N) to phosphours atoms of the nucleic acid (P) to anion moieties of the polymer (A), referred to as the “N:P:A molar ratio”. In certain embodiments, where N:P is from at least 2:1 to no more than 20:1, the molar ratio of P:A can vary from at least 1:40 to about 40:1.

In certain embodiments, where N:P is from at least 2:1 to no more than 20:1, the molar ratio of P:A can vary from at least 1:40 to about 1:10. In certain embodiments, where N:P is from at least 2:1 to no more than 20:1, the molar ratio of P:A can vary from at least 1:25 to about 25:1. In certain embodiments, where N:P is from at least 2:1 to no more than 20:1, the molar ratio of P:A can vary from at least 1:25 to about 1:10. In some cases, where N:P is from at least 2:1 to no more than 20:1, the molar ratio of P:A can vary from at least 1:20 to about 20:1. In some cases, where N:P is from at least 2:1 to no more than 20:1, the molar ratio of P:A can vary from at least 1:20 to about 1:10. In some cases, where N:P is from at least 2:1 to no more than 20:1, the molar ratio of P:A can vary from at least 1:10 to about 10:1. In some cases, where N:P is from at least 2:1 to no more than 20:1, the molar ratio of P:A can vary from at least 1:25 to about 2:1. In some cases, where N:P is from at least 2:1 to no more than 20:1, the molar ratio of P:A can vary from at least 1:20 to about 1:1.

In certain preferred embodiments, N:P:A is from 3:1:3.5 to 3:1:17.5. In certain preferred embodiments, N:P:A is from 5:1:3.5 to 5:1:17.5. In certain preferred embodiments, N:P:A is from 7:1:3.5 to 7:1:17.5. In certain preferred embodiments, N:P:A is from 10:1:10 to 10:1:40. In certain preferred embodiments, N:P:A is about 3:1:3.5, 3:1:7, 3:1:10, 3:1:15, 3:1:17.5, or 3:1:20. In certain preferred embodiments, N:P:A is about 5:1:3.5, 5:1:7, 5:1:10, 5:1:15, 5:1:17.5, or 5:1:20. In certain preferred embodiments, N:P:A is about 7:1:3.5, 7:1:7, 7:1:10, 7:1:15, 7:1:17.5, or 7:1:20. In certain embodiment, N:P:A is about 10:1:10, 10:1:15, 10:1:20, 10:1:25, 10:1:30 or 10:1:40.

1.3.1. Hydrophilic Non-Charged Portion

The hydrophilic non-charged portion of the polymer can be, or comprise, a polyalkylene polyol or a polyalkyleneoxy polyol portion, or combinations thereof. The hydrophilic non-charged portion of the polymer can be, or comprise, a polyalkylene glycol or polyalkyleneoxy glycol portion. In certain embodiments, the polyalkylene glycol portion is or comprises a polyethylene glycol portion and/or a monomethoxy polyethylene glycol portion. In certain preferred embodiments, the non-charged portion of the polymer is, or comprises polyethylene glycol. The hydrophilic non-charged portion of the polymer can be, or comprise, other biologically compatible polymer(s) such as polylactic acid.

In addition to PEG, several hydrophilic non-charged entities are known in the art. For example, see: Lowe et.al., Antibiofouling polymer interfaces: poly(ethyleneglycol) and other promising candidates, Polym. Chem., 6, 198-212, 2015, and Knop et.al., Poly(ethylene glycol) in Drug Delivery: Pros and Cons as Well as Potential Alternatives. Angewandte Chemie International Edition, 49(36), 6288-6308, 2010. Examples of hydrophilic non-charged portion of the polymer are but not limited to: poly(glycerol), poly(2-methacryloyloxyethyl phosphorylcholine), poly(sulfobetaine methacrylate), and poly(carboxybetaine methacrylate), poly(2-methyl-2-oxazoline), poly(2-ethyl-2-oxazoline), and poly(vinylpyrrolidone)

The hydrophilic portion can have a weight average molecular weight of from about 500 Da to about 50,000 Da. In some embodiments, the hydrophilic portion has a weight average molecular weight of from about 1,000 Da to about 10,000 Da. In certain embodiments, the hydrophilic portion has a weight average molecular weight of from about 1,500 Da to about 7,500 Da. In certain embodiments, the hydrophilic portion has a weight average molecular weight of from about 3,000 Da to about 5,000 Da. In some cases, the hydrophilic portion has a weight average molecular weight of, or of about, 5,000 Da.

1.3.2. Anionic Polymer Portion

The anionic polymer portion of the polymer can comprise a plurality of functional groups that are negatively charged at physiological pH. A wide variety of anionic polymers are suitable for use in the methods and compositions described herein, provided that such anionic polymers can be provided as a component of a polymer having a hydrophilic non-charged polymer portion and are capable of forming a (e.g., reversible) charge:charge complex with the positively charged (e.g., dually) derivatized-chitosan-nucleic acid nanoparticles.

Exemplary anionic polymers include, but are not limited to, polypeptides having a net negative charge at physiological pH. In some cases, the polyepeptides, or a portion thereof, consist of amino acids having a negatively charged side-chain at physiological pH. For example, the anionic polymer portion of the polymer can be a polyglutamate polypeptide, a polyaspartate polypeptide, or a mixture thereof. Additional amino acids, or mimetics thereof, can be incorporated into the polyanionic polypeptide. For example, glycine and/or serine amino acids can be incorporated to increase flexibility or reduce secondary structure.

In some cases, the anionic polymers can be or comprise an anionic carbohydrate polymer. Exemplary anionic carbohydrate polymers include, but are not limited to, glycosaminoglycans that are negatively charged at physiological pH. Exemplary anionic glycosaminoglycans include, but are not limited to, chondroitin sulfate, dermatan sulfate, keratin sulfate, heparin, heparin sulfate, hyaluronic acid, or a combination thereof. In certain embodiments, the anionic polymer portion of the polymer is or comprises hyaluronic acid.

Additional or alternative anionic carbohydrate polymers can include polymers comprising dextran sulfate.

In some cases, the polyanion portion is, or comprises, a polyanion selected from the group consisting of polymethacrylic acid and its salts, polyacrylic acid and its salts, copolymers of methacrylic acids and its salts, and copolymers of acrylic acid and/or methacrylic acid and its salts, such as a polyalkylene oxide, polyacrylic acid copolymer.

In some cases, the polyanion portion is, or comprises, a polyanion is selected from the group consisting of alginate, carrageenan, furcellaran, pectin, xanthan, hyaluronic acid, heparin, heparan sulfate, chondroitin sulfate, cellulose, oxidized cellulose, carboxymethyl celluose, crosmarmelose, syntheic polymers and copolymers containing pendant carboxyl groups, phosphate groups or sulphate groups, polyaminoacids of predominantly negative charge, and biocompatible polyphenolic materials.

The anionic portion of the polymers can have a weight average molecular weight of from about 500 Da to about 5,000 Da. In some embodiments, the anionic portion has a weight average molecular weight of from about 500 Da to about 3,000 Da. In certain embodiments, the anionic portion has a weight average molecular weight of from about 500 Da to about 2,500 Da. In certain embodiments, the anionic portion has a weight average molecular weight of from about 500 Da to about 2,000 Da. In certain embodiments, the anionic portion has a weight average molecular weight of from about 500 Da to about 1,500 Da. In some embodiments, the anionic portion has a weight average molecular weight of from about 1,000 Da to about 5,000 Da. In some embodiments, the anionic portion has a weight average molecular weight of from about 1,000 Da to about 3,000 Da. In certain embodiments, the anionic portion has a weight average molecular weight of from about 1,000 Da to about 2,500 Da. In certain embodiments, the anionic portion has a weight average molecular weight of from about 1,000 Da to about 2,000 Da. In some cases, the aninoic portion has a weight average molecular weight of, or of about, 1,500 Da.

As used herein, “block copolymer”, “block co-polymer”, and the like refers to a copolymer containing distinct homopolymer regions. A diblock copolymer contains two distinct homopolymer regions. A triblock copolymer contains three distinct homopolymer regions. The three distinct regions can each be different (e.g., AAAA-BBBB-CCCC), or two regions can be the same (e.g., AAAA-BBBB-AAAA) similar (e.g., AAAA-BBBB-AAA), wherein “A”, “B”, and “C” represent different monomer subunits that form copolymer is comprised. For example, “A” can represent an ethylene glycol monomer subunit of a polyethylene glycol homopolymer and B can represent a glutamic acid subunit of a polyglutamic acid homopolymer. The block copolymer can be a linear (e.g., di- or tri-) block copolymer. Exemplary embodiments of linear diblock and triblock copolymers for use in the subject invention include those listed in Table 2, which is a non-exhaustive list.

TABLE 2
PEG-Polyglutamic acid
methoxy-poly(ethylene glycol)-block-
poly(L-glutamic acid) mPEG*K-b-PLE##
mPEG1K-b-PLE10
mPEG1K-b-PLE50
mPEG1K-b-PLE100
mPEG1K-b-PLE200
mPEG5K-b-PLE10
mPEG5K-b-PLE50
mPEG5K-b-PLE100
mPEG5K-b-PLE200
mPEG10K-b-PLE10
mPEG10K-b-PLE50
mPEG10K-b-PLE100
mPEG10K-b-PLE200
mPEG20K-b-PLE10
mPEG20K-b-PLE50
mPEG20K-b-PLE100
mPEG20K-b-PLE200
PEG-Polyaspartic acid
methoxy-poly(ethylene glycol)-block-
poly(L-aspartic acid) mPEG*K-b-PLD##
mPEG1K-b-PLD10
mPEG1K-b-PLD50
mPEG1K-b-PLD100
mPEG1K-b-PLD200
mPEG5K-b-PLD10
mPEG5K-b-PLD50
mPEG5K-b-PLD100
mPEG5K-b-PLD200
mPEG20K-b-PLD10
mPEG20K-b-PLD50
mPEG20K-b-PLD100
mPEG20K-b-PLD200
PGA-PEG-PGA
poly(L-glutamic acid)-block-poly(ethylene glycol)-
block-poly(L-glutamic acid) PLE##-b-PEG*K-b-PLE##
PLE10-b-PEG1K-b-PLE10
PLE50-b-PEG1K-b-PLE50
PLE100-b-PEG1K-b-PLE100
PLE10-b-PEG5K-b-PLE10
PLE50-b-PEG5K-b-PLE50
PLE100-b-PEG5K-b-PLE100
Polyaspartic-PEG-polyaspartic
poly(L-aspartic acid)-block-poly(ethylene glycol)-
block-poly(L-aspartic acid) PLD##-b-PEG*K-b-PLD##
PLD10-b-PEG1K-b-PLD10
PLD50-b-PEG1K-b-PLD50
PLD100-b-PEG1K-b-PLD100
PLD10-b-PEG5K-b-PLD10
PLD50-b-PEG5K-b-PLD50
PLD100-b-PEG5K-b-PLD100
PEG-poly glutamic acid-PEG
Methoxy-poly(ethylene glycol)-block-poly(L-glutamic
acid)-block-poly(ethylene glycol)
PEG*K-b-PGA##-b-PEG*K
PEG1K-b-PGA10-b-PEG1K
PEG1K-b-PGA50-b-PEG1K
PEG1K-b-PGA100-b-PEG1K
PEG5K-b-PGA10-b-PEG5K
PEG5K-b-PGA50-b-PEG5K
PEG5K-b-PGA100-b-PEG5K
PEG-polyaspartic-PEG
Methoxy-poly(ethylene glycol)-block-
poly(L-aspartic acid)-block-poly(ethylene glycol)
PEG*K-b-PLD##-b-PEG*K
PEG1K-b-PLD10-b-PEG1K
PEG1K-b-PLD50-b-PEG1K
PEG1K-b-PLD100-b-PEG1K
PEG5K-b-PLD10-b-PEG5K
PEG5K-b-PLD50-b-PEG5K
PEG5K-b-PLD100-b-PEG5K
*K: molecular weight of PEG in kDa
## number of subunits

In one embodiment, the block copolymer is or comprises a PEG-polyglutamic acid polymer having the following structure:

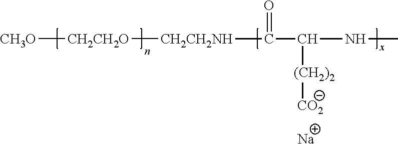

In one embodiment, the block copolymer is or comprises a PEG-polyaspartic acid polymer having the following structure:

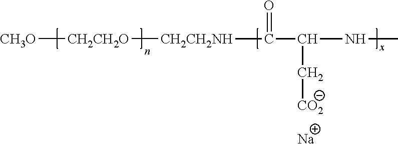

In one embodiment, the block copolymer is or comprises a PEG-hyaluronic acid polymer having the following structure:

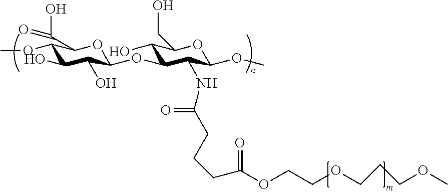

1.4.Methods of Making

As described above, one of skill in the art will appreciate that polyplex:polymer particles of the invention may be produced by a variety of methods. For example, polyplex particles can be generated and then contacted with polymer. In an exemplary non-limiting embodiment, polyplex particles are prepared by providing and combining functionalized chitosan and nucleotide feedstock. Feedstock concentrations may be adjusted to accommodate various amino-to-phosphate ratios (N/P), mixing ratios and target nucleotide concentrations. In some embodiments, particularly small batches, e.g., batches under 2 mL, the functionalized chitosan and nucleotide feedstocks may be mixed by slowly dripping the nucleotide feedstock into the functionalized chitosan feedstock while vortexing the container. In other embodiments, the functionalized chitosan and nucleotide feedstocks may be mixed by in-line mixing the two fluid streams. In other embodiments, the resulting polyplex dispersion may be concentrated by means known in the art such as ultrafiltration (e.g., tangential flow filtration (TFF)), or solvent evaporation (e.g., lyopholization or spray drying). A preferred method for polyplex formation is disclosed in WO 2009/039657, which is expressly incorporated herein in its entirety by reference.

Similarly, polyplex particle feedstock (e.g., an aqueous solution comprising the polyplex compositions) can be provided (e.g., isolated from the reaction mixtures described above) and combined with polymer feedstock (e.g., an aqueous solution comprising the polymer). Feedstock concentrations may be adjusted to accommodate various amino-to-anion ratios (N/A), amino-to-phosphorous (N:P) ratios, N:P:A ratios, mixing ratios and target nucleotide concentrations. In some embodiments, particularly small batches, e.g., batches under 2 mL, the feedstocks may be mixed by slowly dripping a first feedstock (e.g., polyplex) into a second feedstock (e.g., polymer) while vortexing the container. In other embodiments, the feedstocks may be mixed by in-line mixing the two fluid streams. In other embodiments, the resulting polyplex:polymer complex dispersion may be concentrated by means known in the art such as ultrafiltration (e.g., tangential flow filtration (TFF)), or solvent evaporation (e.g., lyopholization or spray drying).

2. Powdered Formulations

The polyplex:polymer compositions of the invention include powders. In a preferred embodiment, the invention provides a dry powder polyplex:polymer composition. In a preferred embodiment, the dry powder polyplex:polymer composition is produced through the dehydration (e.g., spray drying or lyopholization) of a chitosan-nucleic acid polyplex dispersion of the invention.

3. Pharmaceutical Formulations

The present invention also provides “pharmaceutically acceptable” or “physiologically acceptable” formulations comprising polyplex:polymer compositions of the invention. Such formulations can be administered in vivo to a subject in order to practice treatment methods.

As used herein, the terms “pharmaceutically acceptable” and “physiologically acceptable” refer to carriers, diluents, excipients and the like that can be administered to a subject, preferably without producing excessive adverse side-effects (e.g., nausea, abdominal pain, headaches, etc.). Such preparations for administration include sterile aqueous or non-aqueous solutions, suspensions, and emulsions. Liquid formulations include suspensions, solutions, syrups and elixirs. Liquid formulations may be prepared by the reconstitution of a solid.

Pharmaceutical formulations can be made from carriers, diluents, excipients, solvents, dispersion media, coatings, antibacterial and antifungal agents, isotonic and absorption delaying agents, and the like, compatible with administration to a subject. Such formulations can be contained in a tablet (coated or uncoated), capsule (hard or soft), microbead, emulsion, powder, granule, crystal, suspension, syrup or elixir. Supplementary active compounds and preservatives, among other additives, may also be present, for example, antimicrobials, mucolytic agents, anti-oxidants, chelating agents, and inert gases and the like.

Excipients can include a salt, an isotonic agent, a serum protein, a buffer or other pH-controlling agent, an anti-oxidant, a thickener, an uncharged polymer, a preservative or a cryoprotectant. Excipients used in compositions of the invention may further include an isotonic agent and a buffer or other pH-controlling agent. These excipients may be added for the attainment of preferred ranges of pH (about 6.0-8.0) and osmolarity (about 50-400 mmol/L). Examples of suitable buffers are acetate, borate, carbonate, citrate, phosphate and sulfonated organic molecule buffer. Such buffers may be present in a composition in concentrations from 0.01 to 1.0% (w/v). An isotonic agent may be selected from any of those known in the art, e.g. mannitol, dextrose, glucose and sodium chloride, or other electrolytes. Preferably, the isotonic agent is glucose or sodium chloride. The isotonic agents may be used in amounts that impart to the composition the same or a similar osmotic pressure as that of the biological environment into which it is introduced. The concentration of isotonic agent in the composition will depend upon the nature of the particular isotonic agent used and may range from about 0.1 to 10%. When glucose is used, it is preferably used in a concentration of from 1 to 5% w/v, more particularly 5% w/v. When the isotonic agent is sodium chloride, it is preferably employed in amounts of up to 1% w/v, in particular 0.9% w/v. The compositions of the invention may further contain a preservative. Examples preservatives are polyhexamethylene-biguanidine, benzalkonium chloride, stabilized oxychloro complexes (such as those known as Purite®), phenylmercuric acetate, chlorobutanol, sorbic acid, chlorhexidine, benzyl alcohol, parabens, and thimerosal. Typically, such preservatives are present at concentrations from about 0.001 to 1.0%. Furthermore, the compositions of the invention may also contain a cryopreservative agent. Preferred cryopreservatives are glucose, sucrose, mannitol, lactose, trehalose, sorbitol, colloidal silicon dioxide, dextran of molecular weight preferable below 100,000 g/mol, glycerol, and polyethylene glycols of molecular weights below 100,000 g/mol or mixtures thereof. Most preferred are glucose, trehalose and polyethylene glycol. Typically, such cryopreservatives are present at concentrations from about 0.01 to 10%.

A pharmaceutical formulation can be formulated to be compatible with its intended route of administration. For example, for oral administration, a composition can be incorporated with excipients and used in the form of tablets, troches, capsules, e.g., gelatin capsules, or coatings, e.g., enteric coatings (Eudragit® or Sureteric®). Pharmaceutically compatible binding agents, and/or adjuvant materials can be included in oral formulations. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or other stearates; a glidant such as colloidal silicon dioxide; a sweetening agent such as sucrose or saccharin; or a flavoring agent such as peppermint, methyl salicylate, or flavoring.

Formulations can also include carriers to protect the composition against rapid degradation or elimination from the body, such as a controlled release formulation, including implants and microencapsulated delivery systems. For example, a time delay material such as glyceryl monostearate or glyceryl stearate alone, or in combination with a wax, may be employed.

Suppositories and other rectally administrable formulations (e.g., those administrable by enema) are also contemplated. Further regarding rectal delivery, see, for example, Song et al., Mucosal drug delivery: membranes, methodologies, and applications, Crit. Rev. Ther. Drug. Carrier Syst., 21:195-256, 2004; Wearley, Recent progress in protein and peptide delivery by noninvasive routes, Crit. Rev. Ther. Drug. Carrier Syst., 8:331-394, 1991.

Additional pharmaceutical formulations appropriate for administration are known in the art and are applicable in the methods and compositions of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993)).

4. Administration

In one embodiment, the use of polyplexes:polymer compositions provides for prolonged stability of polyplexes at physiological pH. This provides for effective mucosal administration.

Any of a number of administration routes to contact mucosal cells or tissue are possible and the choice of a particular route will in part depend on the target mucosal cell or tissue. Syringes, endoscopes, cannulas, intubation tubes, catheters and other articles may be used for administration.

The doses or “effective amount” for treating a subject are preferably sufficient to ameliorate one, several or all of the symptoms of the condition, to a measurable or detectable extent, although preventing or inhibiting a progression or worsening of the disorder or condition, or a symptom, is a satisfactory outcome. Thus, in the case of a condition or disorder treatable by expressing a therapeutic nucleic acid in target tissue, the amount of therapeutic RNA or therapeutic protein produced to ameliorate a condition treatable by a method of the invention will depend on the condition and the desired outcome and can be readily ascertained by the skilled artisan. Appropriate amounts will depend upon the condition treated, the therapeutic effect desired, as well as the individual subject (e.g., the bioavailability within the subject, gender, age, etc.). The effective amount can be ascertained by measuring relevant physiological effects.

Veterinary applications are also contemplated by the present invention. Accordingly, in one embodiment, the invention provides methods of treating non-human mammals, which involve administering a polyplex:polymer composition of the invention to a non-human mammal in need of treatment.

4.1 Mucosal Administration

The compositions of the invention may be administered to the mucosa. For example, target mucosal cells or tissues include, but are not limited to airway epithelial and lung cells.

In preferred embodiments, the compositions and formulations of the invention are administered to the airway epithelium by inhalation, typically in the form of a dry powder (either alone, as a mixture, for example, in a dry blend with lactose, or as a mixed component particle) from a dry powder inhaler or as an aerosol spray from a pressurized container, pump, spray, atomiser, or nebuliser, with or without the use of a suitable propellant.

Formulations for inhaled/intranasal administration may be formulated to be immediate and/or modified release. Modified release formulations include delayed-, sustained-, pulsed-, controlled-, targeted and programmed release.

Nebulizers are a preferred method for pulmonary drug delivery, whereby drug solution is aerosolized and inhaled for direct delivery to lung tissue. Mechanical or electrical nebulizers, including soft mist inhalers, jet nebulizers, ultrasonic wave nebulizers or vibrating mesh nebulizers may be used to deliver the compounds of the invention. Also contemplated for use herein are more conventional metered dose inhalers employing, e.g., CFCs (Chlorofluorocarbons) or HFA (hydrofluoroalkane) as the propellant.

Vibrating mesh nebulizers offer several advantages over other nebulizer types such as higher output rate, shorter treatment time, and less drug degradation (lower shear stress). As one example, Aerogen has developed two different vibrating mesh technologies: (1) a commercially available single-use mesh nebulizer (Aerogen Solo) and (2) a novel next-generation Photo Defined Aperture Plate (PDAP) technology by Aerogen Pharma. The novel PDAP device was designed to reduce droplet size and increase relative lung dose efficiencies.

Other vibrating mesh nebulizers for use delivering the compounds described herein to a patient in need thereof include, but are not limited to the Mobi Mesh (Apex), M-NEB FLOW+ (Salvia), Prodigy MiniMist®, Mexus BBU01, Hannox MA-02, K-Jump KN-9100, Quatek NM211, Health & Life HL100A, KTMed Inc. NEPLUS (NE-SM1), B.Well WN-114, DigiO2 0.3 ml/min DIGIO2®, Pari Veliox, and the OK Biotech Docspray.

Capsules, blisters and cartridges for use in an inhaler or insufflator may be formulated to contain a powder mix of the compound of the invention, a suitable powder base such as lactose or starch and a performance modifier such as I-leucine, mannitol, or magnesium stearate.

In some embodiments, typical formulations for delivery of the compounds of the invention include liquids, gels, hydrogels, solutions, creams, foams, films, implants, sponges, fibres, powders, and microemulsions.

### Therapeutic Applications

Therapeutic proteins contemplated for use in the invention have a wide variety of activities and find use in the treatment of a variety of lung disorders. The following description of therapeutic protein activities, and indications treatable with therapeutic proteins and therapeutic RNA of the invention, is exemplary and not intended to be exhaustive. The term “subject” refers to an animal, with mammals being preferred, and humans being especially preferred. Specific non-limiting examples of therapeutic embodiments are described below. In some cases, the therapeutic embodiments are intended to act on non-mucosal target tissues, cells, or organs. Where the therapeutic effect is non-mucosal, it is understood that the cells or tissues contacted by the polyplex:polymer compositions described herein are mucosal and the therapeutic action is distal to the mucosal target. For example, mucosal cells can be transfected to produce and secrete a hormone or other therapeutic molecule.

In one embodiment, polyplex:polymer compositions of the invention may be used for therapeutic treatment. Such compositions are sometimes referred to herein as therapeutic compositions.

A partial list of therapeutic proteins and target diseases is shown in Table 3.

| Lung Disorders | Therapeutic Protein or RNA Target |
|---|---|
| Cystic fibrosis | Cystic Fibrosis Transmembrane |
| | Condutance Regulator (CFTR) |
| Alpha-1 antitrypsin | Alpha-1 antitrypsin |
| Deficiency | |
| Idiopathic pulmonary | Fibroblast growth factor (FGF-1, FGF-7, FGF-10) |
| fibrosis | Inhibitory antibodies to TGFb1, PDGF or avb6 integrin |
| | Surfactant-associated proteins |
| | Telomerase reverse transcriptase (TERT) |
| | siRNA to TGFb1 |
| | Hepatocyte growth factor (HGF) |
| | MicroRNAs hsa-miR 17-92 and let-7d |
| | Antisense, shRNA or siRNA to mutated SFTA protein C |
| | PTEN-induced putative kinase 1 (PINK1) |
| | Angiotensin-converting enzyme 2 (ACE2) |
| | IL-22 |
| Lung cancer | Agonists of pattern recognition receptors |
| | (TLR, STING and RIG-I) |
| | IL-12 |
| | CXCL10 |
| | CXCL9 |
| | IL-15 |
| | OX40L |
| | CD40L |
| | 4-1BBL |
| | Antibody or fragment thereof (e.g. scFv) to CD40 |
| | Antibody or fragment thereof to CD47 |
| | SIRP1alpha |
| | GM-CSF |
| | Inhibitory antibody or fragment thereof to TGFb1 |
| | Inhibitory antibody or fragment thereof |
| | to PD-L1 and CTLA-4 |
| | PD-1 |
| | Antibody or fragment thereof to CD39 |
| | Antibody or fragment thereof to CD73 |
| Asthma | IL-10 |
| | Thymulin |
| | Antibody or fragment thereof to IL-18 or IL-5 |
| | Antisense, shRNA or siRNA to IL-5, IL-4, |
| | CD86, CD40 or IL-4Ra |
| | Antibody or fragment thereof to IL-13 |
| | Fc-coupled soluble ST-2 |
| | Antibody or fragment thereof to ST2 |
| | Antibody or fragment thereof to IL-33 |
| | Antibody or fragment thereof to TSLP |
| | IL-12 |
| | IL-1Ra |
| | IL-37 |
| Chronic obstructive | Antibody or fragment thereof to IL-18 |
| pulmonary disease | IL-18 binding protein |
| (COPD) | Antisense, siRNA or shRNA to Caspase-1 |
| | Antisense, shRNA or siRNA to |
| | CXCL1 or CXCL2 (MIP-2) |
| | Antibody or fragment thereof to CXCR2 |
| | Secretory leukoprotease inhibitor (SLPI) |
| | Alpha-1 antitrypsin |
| | Antibody or fragment thereof to TGF-B1 |
| Acute Respiratory | Antibody or fragment thereof to IL-6 |
| Distress Syndrome | NFkb decoy |
| (ARDS) or | IL-10 |
| Acute Lung Injury | Angiopoetin-1 |
| (ALI) | HSP-70 |
| | IL-1Ra |
| | Alpha-1 antitrypsin |
| Pulmonary arterial | BMPR2 |
| hypertension (PAH) | BMP9 |
| | SOX17 |
| | siRNA, shRNA or antisense to HDAC |
| | siRNA, shRNA or antisense PARP1 |
| SARS-COV2 | Antibody or fragment thereof to ACE2 |
| | ACE2 decoy |
| | Antibody or fragment thereof to spike protein |
| | Antibody or fragment thereof to spike RBD |

Therapeutic nucleic acids of particular interest encode functional CFTR or functional hAAT.

### Methods of Treatment

### Cystic Fibrosis Pulmonary Function

The subject compositions and methods find advantageous use in the treatment of ion channel disorders of mucosal tissues or in tissues proximal to mucosal tissue. Of particular interest is the treatment of cystic fibrosis and related disorders, including the treatment of CF patients who are non-responsive to existing therapies. The subject compositions and methods may also find advantageous use in combination with existing therapies, by improving efficacy and outcomes in patients who are responsive or only partially responsive to existing therapies.

In some embodiments, the compositions and methods described herein are used for the treatment of CF in patients who are non-responsive to current therapies. In an exemplary embodiment, the subject compositions and methods find advantageous use in the treatment of CF in patients having a null mutation in the CFTR gene, e.g., one or more Class 1 mutations.

In some embodiments, the compositions and methods described herein can be used as part of a combination therapy with another cystic fibrosis cystic drug including, but not limited to a CFTR potentiators, such as ivacaftor (Kalydeco®), CFTR correctors include lumacaftor/ivacaftor (marketed as Orkambi®), tezacaftor/ivacaftor (marketed as Symdeko®), and elexacaftor/tezacaftor/ivacaftor (Trikafta™) produced by Vertex Pharmaceuticals, or a CFTR stabilizer, such as cavosonstat (N91115), to improve efficacy and/or reduce toxicity in responsive or partially responsive patients.

In some embodiments, the compositions and methods described herein can be used for the treatment of CF in an individual having a Class 1 CFTR mutation. In some embodiments the compositions and methods described herein can be used for the treatment of CF in an individual having a Class 2 or Class III CFTR mutation. In some embodiments, the compositions and methods described herein can be used for the treatment of CF in an individual having a Class 4 or 5 or 6 mutation, preferably in combination with one or more existing CF therapies.

In preferred embodiments, the compositions described herein can be delivered via a nebulizer for the treatment of cystic fibrosis.

In some embodiments, the compositions and methods as described herein are used in combination with anti-inflammatory gene therapy. In some embodiments, the CFTR gene and at least one anti-inflammatory gene are carried in separate plasmids. In some embodiments the CFTR gene and at least one anti-inflammatory gene are contained within the same plasmid.

In some embodiments, the compounds as described herein are used in combination with conventional anti-inflammatory agents such as, but not limited to a steroid or steroid-like agent, such as a corticosteroid, budesornde, beclomethasone, fluticasone, or flunisolide.

In some embodiments, a compound as described herein is used in combination with an existing supportive therapy or therapies for cystic fibrosis including, but are not limited to, oral or I.V. antibiotics including penicillins such as amoxicillin and clavulanic acid (Augmenting), dicloxacillin, Nafcillin and oxacillin, piperacillin/tazobactam (Zosyn®), cephalosporins including cephalexin, cefdinir, cefutoxime, ceftazidime, ceftazidime/avibactam, cefepime, cefoxitin, ceftolozane-tazobactam, ceftaroline, and cefazolin, carbapenems including meropenem, imipenem/cilastin, doripenem, meropenem-avibactam, and ertapenem, sulfa drugs including sulfamethoxazole and trimethoprim (Bactrim®), tetracyclines including tetracycline, doxycycline, minocycline, and tigecycline, vancomycin, clindamycin, linezolid, aminoglycosides including tobramycin, amikacin, and gentimicin, macrolides including azithromycin, quinolones including ciprofloxacin, moxifloxacin and levofloxacin; and others including aztreonam, Colistin®, rifampin, clindamycin, mupirocin, clofazimine, co-trimoxazole, levofloxacin, and ethambutol. In some embodiments, the supportive therapy includes treatment with one or more of the following: an airway surface hydrators, one or more mucolytics including DNase, Guaifenesin, N-acetylcysteine, hypertonic saline and dornase alfa (Pulmozyme®), inhaled antimicrobials, a bronchodilator such as albuterol, theophylline, or ipratropium, a systemic anti-inflammatory treatments such as Triamcinolone, Flunisolide, Fluticasone, Beclomethasone, Prednisone, Methylprednisone, Ibuprofen, Montelukast, Cromolyn, N-acetylcysteine, vitamins including, but not limited to ADEK, Fer-inSol, polyviflor drops, Aquasol-A, Drisdol, Aquasol-E, pancreatic enzymes including pancrelipase, and pancreatin, a stool softened such as docusate, casanthranol/Docusate, or polyethylene glycol, a drug for relief of GI reflux such as omeprazole, rantidine, or metoclopramide, an antihistamine such as loratadine, cetririzine or fexofenadine, a nasal spray such as Vancenase/vane; AQ, Beconase/bexAq or a sinus rinse and/or nutritional support. In some cases lung transplants are performed.

### COPD Pulmonary Function

The subject compositions and methods find advantageous use in the treatment of chronic inflammatory lung diseases causing obstructed airflow in the lungs. Of particular interest is the treatment of pulmonary disease related to the presence of a mutated alpha-1 anti-trypin gene (hAAT). The subject compositions and methods may also find advantageous use in combination with existing therapies, by improving efficacy and outcomes in patients who are responsive or only partially responsive to existing therapies.

In some embodiments, the compositions and methods described herein are used for the treatment of CF in patients who are non-responsive to current therapies.

In some embodiments, the compounds as described herein are used in combination with conventional anti-inflammatory agents such as, but not limited to a steroid or steroid-like agent including, but not limited to prednisone, hydrocortisone, prednisolone, methylprednisolone, and dexamethasone, beclomethasone diproprionate, budesonide, ciclesonide, flunisolide, fluticasone propionate, and mometasone. In some embodiments the steroid or steroid-like agent is taken orally In some embodiments the steroid or steroid-like agent is inhaled.

The examples set out herein illustrate several embodiments of the present disclosure but should not be construed as limiting the scope of the present disclosure in any manner.

### EXAMPLES

### Example 1

Measure of Plasmid Transfection Efficacy In Vitro.

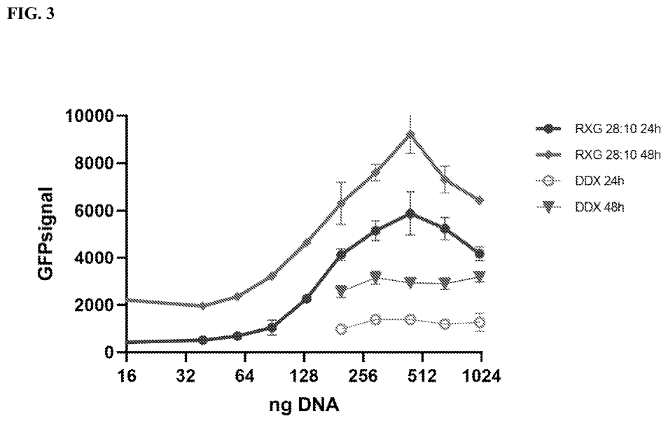

Human bronchial epithelial (HBE) cells were seeded in 96 well plates (27,000 cells/well) 24 hours prior to transfection with GFP expression vectors (NTC9385R-eGFP) using RXG or DDX polyplex carriers. The unpegylated polyplexes containing GFP expressing vectors were used at increasing doses of plasmid DNA to transfect HBE. At 24 and 48 hours post-transfection, the GFP fluorescence intensity was examined using a plate reader (EnVision; PerkinElmer). As seen in FIG. 3, RXG 28:10 polyplex transfects HBE cells in a dose-dependent manner.

### Example 2

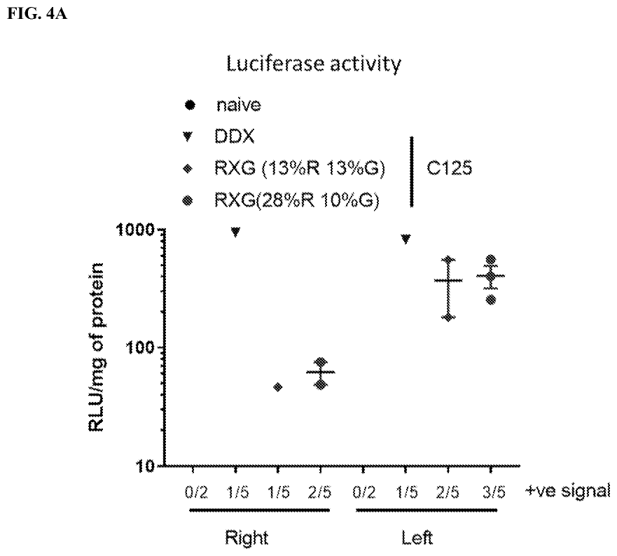

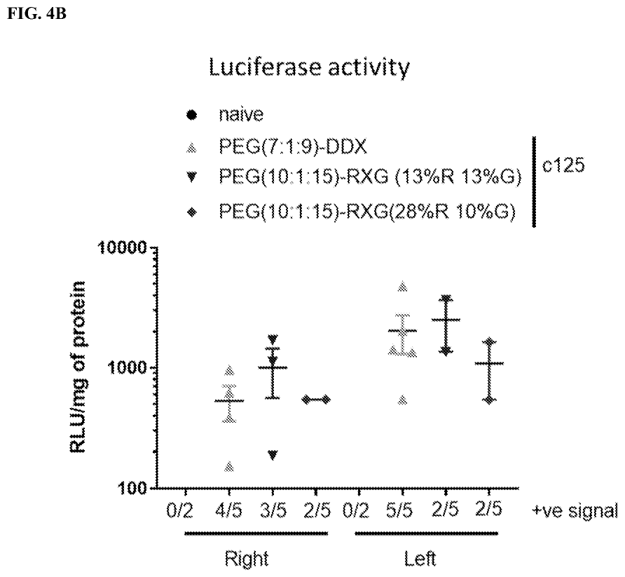

Enzyme activity of Luciferase reporter gene (Luc2) following intranasal instillation of unpegylated and pegylated formulations. Male mice were anesthetized using isoflurane. Animals were then scruffed and their heads were given a slight angled to allow the liquid formulation to slide down the nostril. Using a pipette, 50uL of polyplex were slowly dispensed allowing the animals to aspirate the liquid. At 24 hours post-administration, lungs were harvested, all the right lobes were pooled together in one tube and the left lobe was collected by itself. Lungs were homogenized in cell culture lysis buffer (Promega) and assessed for luciferase activity using Luciferase substrate (Promega) and read on the EnVision (PerkinElmer). Total protein content was measured by Bradford as quantifiable levels of relative light units (RLUs) per mg of protein. As seen in FIG. 4A, both unpegylated DDX and RXG formulations can transfect lungs following intranasal delivery. As seen in FIG. 4B, and FIG. 4B, pegylated DDX and RXG formulations lead to quantifiable luciferase activity in lungs following intranasal delivery.

### Example 3

Duration of Expression In Vivo

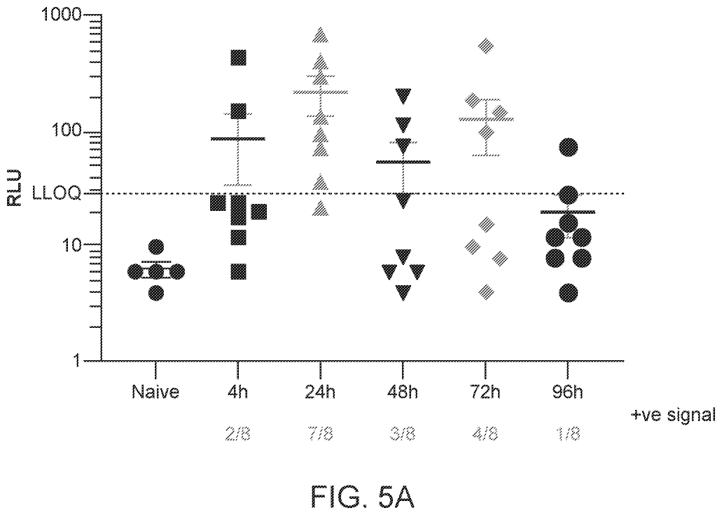

As in previous studies, male mice were anesthetized using isoflurane. Animals were then scruffed and their heads were given a slight angled to allow the liquid formulation to slide down the nostril. Using a pipette, 50uL of the C125 pegylated DDX (7:1:9) were slowly dispensed allowing the animals to aspirate the liquid. At the indicated timepoints post-administration, lungs were harvested, the right lobes were pooled together in one tube and the left lung was collected in a second tube. Half of each group was processed for luciferase activity and the other half for RNA extraction. For luciferase activity (top), lungs were homogenized in cell culture lysis buffer (Promega) and assessed for luciferase activity using Luciferase substrate (Promega) and read on the EnVision (PerkinElmer). As seen in FIG. 5A, data is represented as relative light units (RLUs) per mg of protein. For RTqPCR, tissue was homogenized at collection and RNA was extracted (Qiagen, RNeasy kit). Rt-qPCR was performed using 3ug of input using TaqMAN primer/probe that recognize Luc2. Absolute quantification was performed using a standard curve of Luc2. Data is presented as RNA copy number (FIG. 5B).As can be seen in FIGS. 5A and 5B, detectable and durable luciferase expression and activity were seen in lungs for at least 96 hours post-administration.

### Example 4

Dose Response of Single Intranasal Instillation of Pegylated DDX Polyplex

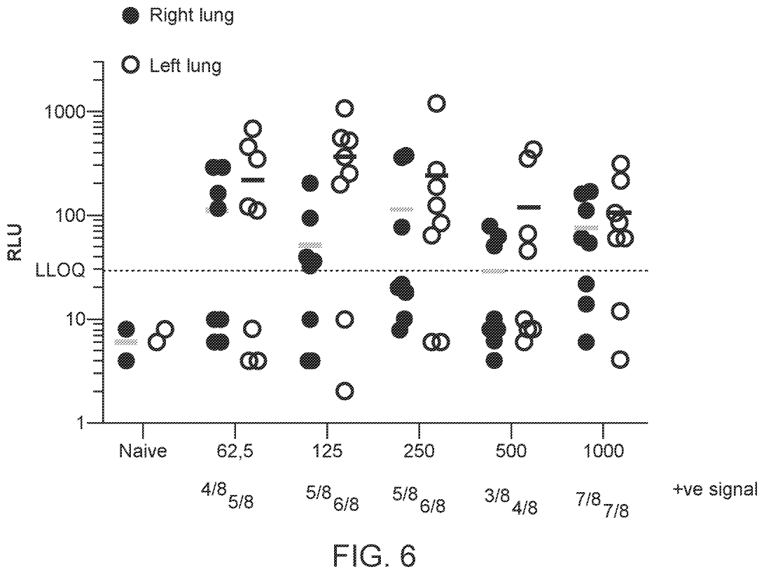

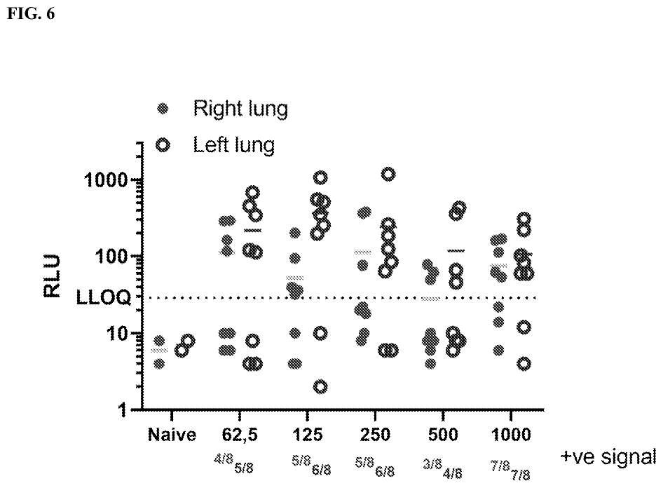

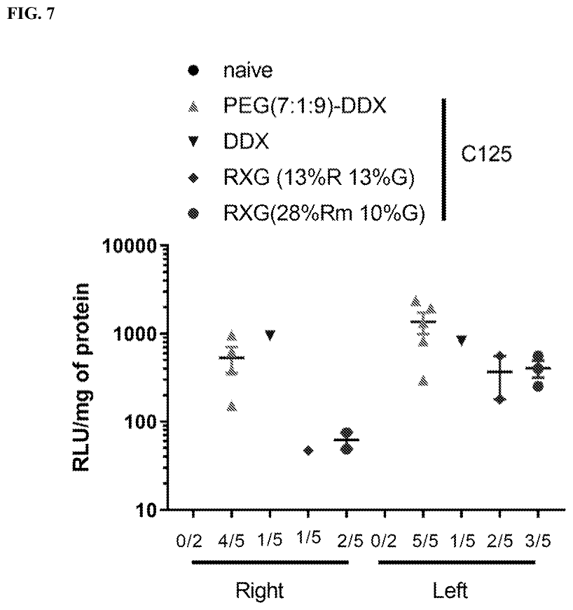

Male mice were anesthetized using isoflurane. Animals were then scruffed and their heads were given a slight angled to allow the liquid formulation to slide down the nostril. Using a pipette, 50 uL of the pegylated DDX (7:1:9) formulation, at the indicated concentrations, were slowly dispensed allowing the animals to aspirate the liquid. At 24 hours post-administration, lungs were harvested, the right lobes were pooled together in one tube and the left lung was collected in a second tube. Lungs were homogenized in cell culture lysis buffer (Promega) and assessed for luciferase activity using Luciferase substrate (Promega) and read on the EnVision (PerkinElmer). Data is represented as relative light units (RLUs). As seen in FIG. 6, pegylated DDX formulations lead to equivalent levels of luciferase activity in murine lungs across a wide range of concentrations.

### Example 5

Preparation of Dually Derivatized Chitosan and DNA Polyplexes for Aerosol Delivery

Chitosan was dually derivatized (DD-chitosan, DD-X) with arginine and gluconic acid according to U.S. Pat. No. 9,623,112 B2. DD-chitosan was polyplexed with a plasmid DNA vector according to U.S. Pat. Nos. 9,623,112B2 and 8,722,646B2 at various amine-to-phosphate (N:P) molar ratios, as required. Additional excipients such as sucrose, trehalose or mannitol were included as required. Various plasmid DNA vectors were tested as indicated herein.

### Reagents

Trehalose was dissolved in water at a concentration of up to 0.2 g/mL as required. The resulting trehalose filtered using a 0.2 um filter. The trehalose solution was diluted to required concentration necessary for subsequent formulations.

A PAA solution in 50 mM Tris, pH 8 was prepared using standard techniques at a concentration of 100 mg/mL or 20 mg/mL as required. If necessary, the PAA solution was diluted in 50 mM Tris pH 8 to required concentration necessary for subsequent need.

20 mM NaCl was made by dissolving 46.75 mg of NaCl in 40 mL of w and filtering through a 0.2 um filter.

### Polyplex Preparation

PEGylated polyplexes were prepared by dripping equal volume of polyplex solution (0.1 mL) into diluted PEG-PA solution (0.1mL) using an in-line mixing apparatus such as described in U.S. Pat. Nos. 9,623,112B2 and 8,722,646B2. Alternatively PEG-PLE was added to the DNA solution and subsequently polyplexed with DD-X to form nanoparticles directly at various amine-to-phosphate (N:P) molar ratios, as required. Additional excipients such as sucrose, trehalose or mannitol were included as required.

### Nebulization of PEGylated Polyplexes in 5% Trehalose

DD-X-DNA (NTC9385R-Luc2) polyplexes were produced at N:P ratio of 7:1 or 10:1 with a DNA concentration of 0.25 mg/mL DNA in 5% trehalose as previously described. The polyplex were then PEGylated (mPEG5K-b-PLE10) as previously described to various NPA ratios at a DNA concentration of 0.125 mg/mL. Some of the polyplexes were then concentrated by TFF to a final DNA concentration of 1 mg/mL. The resulting formulations aliquoted into cryovials and frozen at −80C for future use.

Samples were thawed and loaded (1-2 mL) into the reservoir of the Aerogen Solo nebulizer units. The exit port was directed into a 50 mL conical tube and sealed with parafilm to prevent vapour loss of sample. The Aerogen Solo was connected to the Aerogen Pro-X Controller and the samples were aerosolized by pressing the power button on the controller unit. The nebulized samples were briefly centrifuged to collect the aerosol liquid. Each test sample with and without nebulization treatment were analyzed for particle size, PDI, zeta potential, % supercoil DNA, free DNA, and osmilality as described herein. Test samples were also analyzed for DD-X content, PEG content, and total DNA content to assess recovery of nanoparticle components as compared to original freeze/thaw samples.

Particle size was measured using gel electrophoresis. Osmolality was measured by freezing point depression using a Micro Osmette Osmometer. Samples were analyzed in triplicates and average values were reported with standard deviations (n=3). Nanoparticle sizing of polyplexes and PEGylated polyplexes were made using a Zetasizer Nano light scattering instrument. In general, samples were diluted to 0.125 mg/mL in water and subsequently diluted 8-fold in 10 mM NaCl and loaded into a disposable cuvette or a Zetasizer folded capillary cell (0.8 mL minimum). The Zetasizer was programmed to incubate the sample for up to 3 minutes at 25° C. prior to triplicate 3-minute measurements. Z-average diameter and polydispersity (PDI) were reported with standard deviation (n=3). The Zetasizer was also programmed to account for the composition of the samples with regards to viscosity and refractive index. Zeta potential measurements of polyplexes and PEGylated polyplexes were made using a Zetasizer Nano light scattering instrument. In general, samples were diluted to 0.125 mg/mL in water and subsequently diluted 8-fold in 10 mM NaCl and loaded into a Zetasizer folded capillary cell (0.8 mL minimum). The Zetasizer was programmed to incubate the sample for up to 3 minutes at 25° C. prior to replicate measurements (number of replicates were automatically determined by Zetasizer software). Zeta potential values were reported with standard deviation (n=3). The Zetasizer was also programmed to account for the final composition of the samples with regards to viscosity and dielectric constant.

### Results

All formulations were successfully aerosolized by Aerogen Solo vibrating mesh nebulizer. The appearance, particle size, PDI, zeta potential, % supercoil DNA, free DNA, and osmolality of the test samples pre- and post-nebulization are summarized in Table 3.

TABLE 3

| | Nebulized | | Size | | | ZP | | | Osm. |
|---|---|---|---|---|---|---|---|---|---|
| Entity ID | (Y/N) | Appearance | (nm) | PDI | pH | (mV) | % SC | Free DNA | (mOsm/kg) |
| CMC-INT08-001 B | No | Clear/ | 125 | 0.185 | 6.20 | −0.63 | 90 | No unbound | 203 |
| | | Translucent | | | | | | DNA | |
| | Yes | Clear/ | 127 | 0.182 | n/a | −0.45 | 90 | No unbound | 183 |
| | | Translucent | | | | | | DNA | |
| CMC-INT08-001 F | No | Clear/ | 112 | 0.208 | 6.08 | −0.96 | 92 | No unbound | 179 |
| | | Translucent | | | | | | DNA | |
| | Yes | Clear/ | 110 | 0.207 | n/a | −0.91 | 93 | No unbound | 177 |
| | | Translucent | | | | | | DNA | |
| CMC-INT08-002 | No | Clear/ | 132 | 0.171 | 7.04 | −2.48 | 91 | No unbound | 186 |
| | | Translucent | | | | | | DNA | |
| | Yes | Clear/ | 134 | 0.164 | n/a | −2.49 | 89 | No unbound | 180 |
| | | Translucent | | | | | | DNA | |

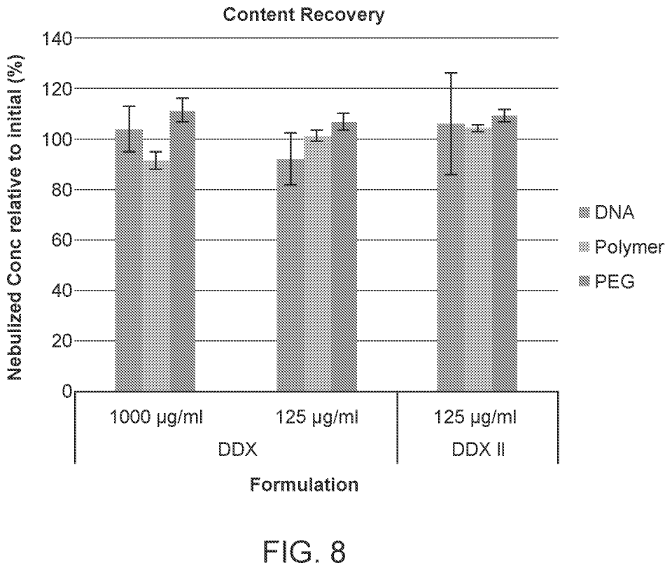

Results showed no change in DD-X, PEG-PLE and DNA (NTC9385R-Luc2) concentration between pre- and post-nebulized test samples, as seen in Table 4. FIG. 8 depicts the recovery of DD-X polymer, PEG-PLE, and DNA content in polyplex formulations after nebulization relative to no nebulization treatment.

TABLE 4

| | Nebulized | [DNA] | | [DD-X] | | | |
|---|---|---|---|---|---|---|---|
| Entity ID | (Y/N) | μg/mL | % Recovery | (mM) | % Recovery | [PEG-PLE] | % Recovery |
| CMC-INT08-001 B | No | 115 | 92 | 2.45 | 102 | 2.80 | 107 |
| | Yes | 106 | | 2.49 | | 2.95 | |
| CMC-INT08-001 F | No | 125 | 106 | 3.48 | 105 | 4.58 | 110 |
| | Yes | 133 | | 3.64 | | 5.02 | |
| CMC-INT08-002 | No | 892 | 104 | 19.71 | 92 | 19.71 | 112 |
| | Yes | 930 | | 18.08 | | 22.09 | |

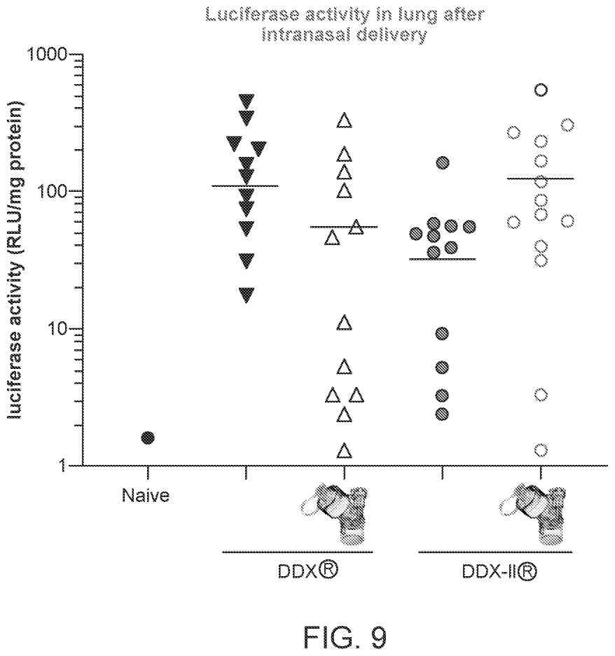

FIG. 9 depicts enzyme activity levels of Luciferase reporter gene activity in the lungs of mice at 24 hour after intranasal delivery of various dispersed polyplexes, containing plasmid DNA packaged with either DDX and RXG, before and after nebulization with a mesh nebulizer. PEGylated polyplexes containing different types of DD-X polymer at DNA concentrations from 0.125 mg/mL-1 mg/mL were successfully aerosolized by passage through vibrating mesh nebulizers without change to colloidal stability and physicochemical properties. All product components were fully recovered and in vivo potency was preserved post-nebulization.

Additionally, the in vivo potency of the polyplexes was preserved following passage through mesh nebulizers.

### Example 6

Transfection of Differentiated Epithelial Cells at an Air Liquid Interface with Pegylated-RXG

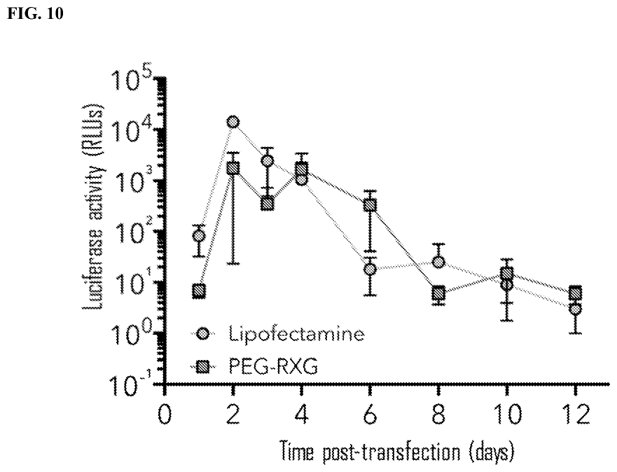

Primary Human Bronchial Epithelial (HBE) from healthy donors were cultured at an air-liquid interface (ALI) in 24-well transwell (70 000 cells/well). At maturation (day 21), HBEs were transfected using with 2 μg RXG-pDNA nanoparticles or lipofectamine that expresses a luciferase reporter gene. Samples were collected at the indicated time points and were lysed using Luciferase Cell Culture Lysis Reagent (1X, Promega). The enzyme substrate luciferin was added to the lysates and bioluminescence was measured on the EnVision plate reader (Perkin-Elmer). As shown in FIG. 10, transfection of NBE cells with RXG-pDNA is sustained for more than 6 days.

### Example 7

Expression Following Intranasal Instillation

Polyplexes were incubated at room temperature for a minimum of 15 minutes and used within 4 h. Male mice (9-11 weeks) were anesthetized by isoflurane and were administered intranasally (IN) with 50 uL of 125 ug DNA/mL of DDX nanoparticle containing luciferase expressing vector. Animals were held in an upright position and the formulation was slowly dispensed on the nostrils. This position was held for 15-30 seconds to ensure proper uptake of the formulation.

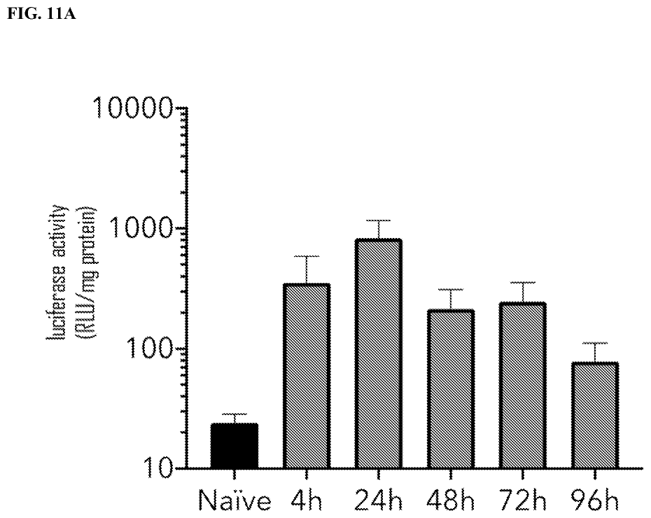

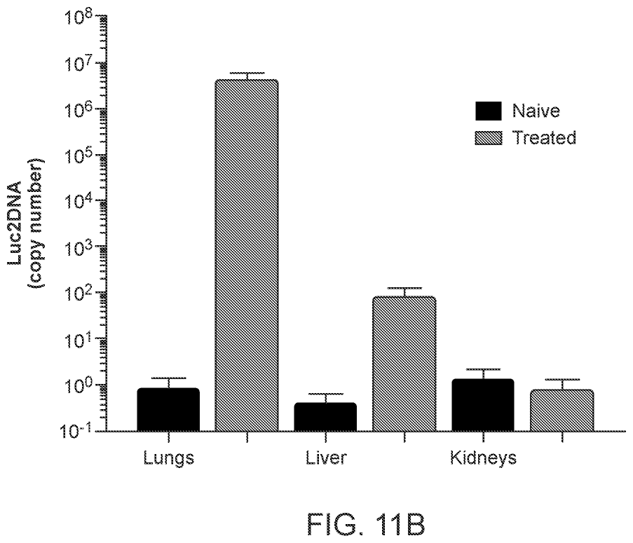

Lungs were collected at the defined timepoints and homogenized in Luciferase Cell Culture Lysis Reagent (1X, Promega). The enzyme substrate luciferin was added to the lysates and bioluminescence was measured on the EnVision plate reader (Perkin-Elmer). A Bradford assay was performed to quantify the total protein in the homogenate. As seen in FIG. 11A, DDX nanoparticles administered intranasally induce durable expression in lungs over a 96 hour period.

Animals were sacrificed 24 hours post-instillation. The lungs, liver and kidneys were homogenized in RLT and the RNA was extracted (Qiagen RNeasy kit). Plasmid DNA levels were quantified by quantitative PCR using 1 ug of input RNA and TaqMan primer/probes that recognize Luc2. Absolute quantification was performed using a standard curve of Luc2 pDNA. Data are represented as DNA copy number. As seen in FIG. 11B, there is elevated expression in lungs, with minimal expression in liver and kidneys, showing that DDX nanoparticles administered intranasally lead to limited systemic exposure.

### Example 8

CFTR Expression Following Single Intratracheal Administration in Mice

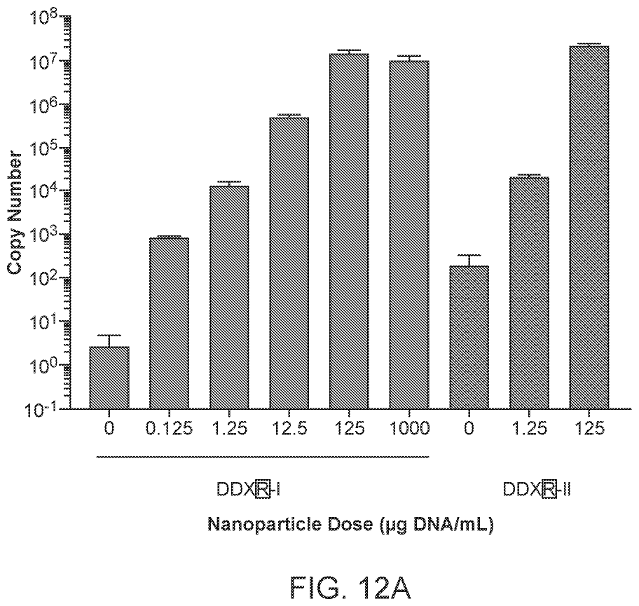

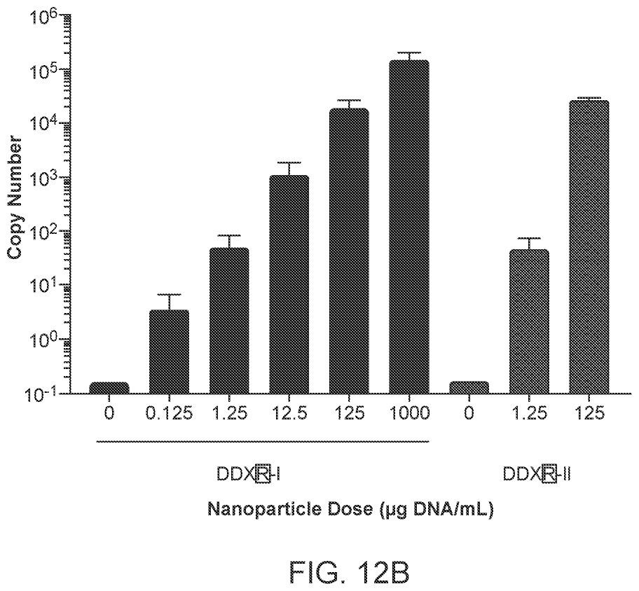

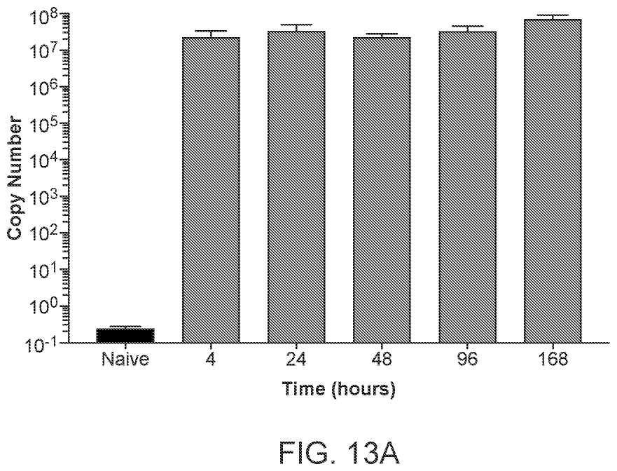

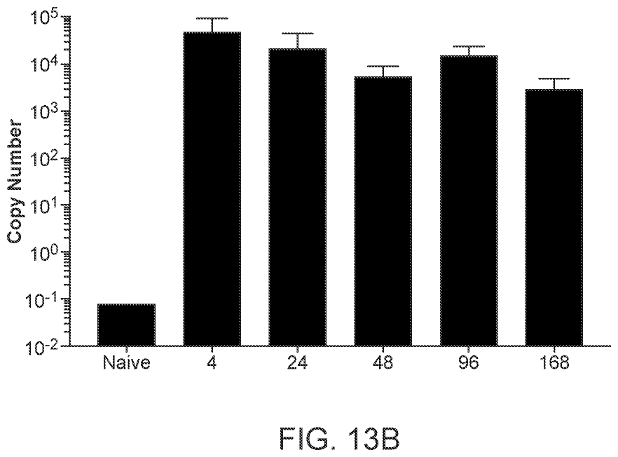

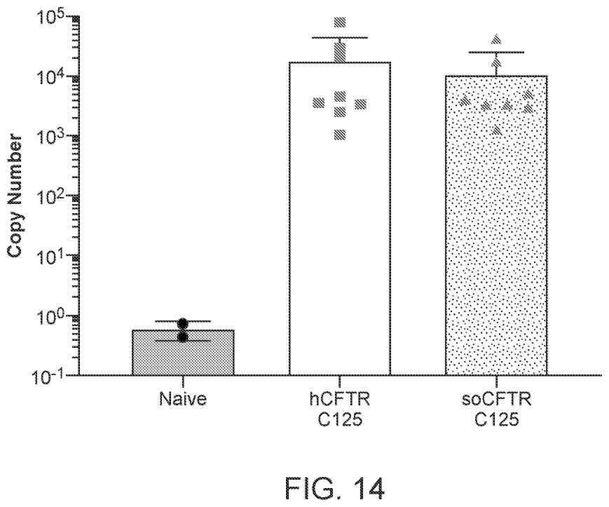

DDX Polyplexes were incubated at room temperature for a minimum of 15 minutes and used within 4 h. Male mice (9-11 weeks) were anesthetized by isoflurane. Once fully anesthetized, animals were transferred to a dosing platform and maintained under low level anesthesia. Catheter placement in the trachea was confirmed by verifying air flow through the catheter. A combination of 50 uL of nanoparticles of Cystic fibrosis transmembrane conductance regulator (CFTR)-containing plasmids (Ref CFTR, or CpG Free CFTR) at 125 μg DNA/mL and 150 uL of air were administered. The catheter was removed, and animals were maintained in an upright position for about 10 seconds to allow dispersal of the liquid. Lungs were collected 48 hours post-instillation, homogenized in RLT and the RNA was extracted (Qiagen RNeasy kit). The expression of mRNA was analyzed by RT-qPCR using TaqMan primer that are specific for hCFTR or soCFTR. Absolute quantification was performed using a standard curve of mRNA. As seen in FIGS. 12A and 12B, there is a dose dependent correlation between delivered nanoparticles (FIG. 12A) and mRNA abundance of target transcript (FIG. 12B), demonstrating robust delivery with intratracheal administration. As seen in FIGS. 13A and 13B, the delivered nanoparticles and mRNA levels of the target transcript remained detectable at elevated levels at least a week after instillation, suggesting that weekly dosage is feasible. As seen in FIG. 14, robust expression of CFTR was seen in mouse lungs 48 hours after a single intratracheal administration.

### Example 9

Distribution of Nanoparticles in the Lung

Polyplexes were incubated at room temperature for a minimum of 15 minutes and used within 4 h. Male mice (9-11 weeks) were anesthetized by isoflurane. Once fully anesthetized, animals were transferred to a dosing platform and maintained under low level anesthesia. Catheter placement in the trachea was confirmed by verifying air flow through the catheter. A combination of 50 uL of nanoparticles of Luc2-containing plasmids at 125μg DNA/mL and 150 uL of air was administered. Following catheter removal, animals were maintained in an upright position for about 10 seconds to allow dispersal of the liquid. 48 hours post-instillation, the lungs were perfused with 500uL of 10% buffered formalin and maintained in 10% buffered formalin for 24 hours. Samples were transferred to 70% ethanol and were embedded in paraffin and sectioned.

Fluorescence in situ hybridization (FISH) performed using RNAscope (ACD) with probes that recognize the DNA and mRNA of the Luc2 vector (red) and cell nuclei were stained by DAPI (blue) showed distribution of the nanoparticles throughout the lungs of tested animals

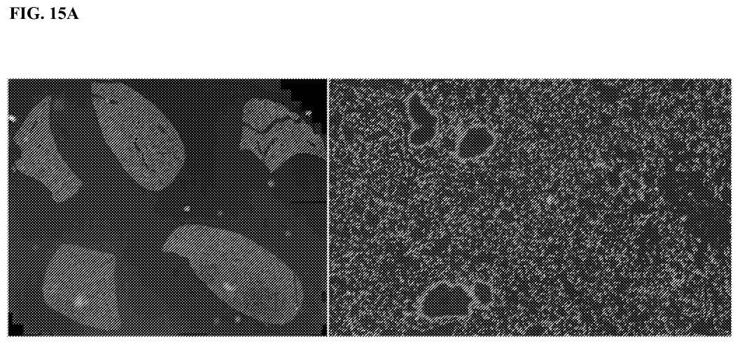

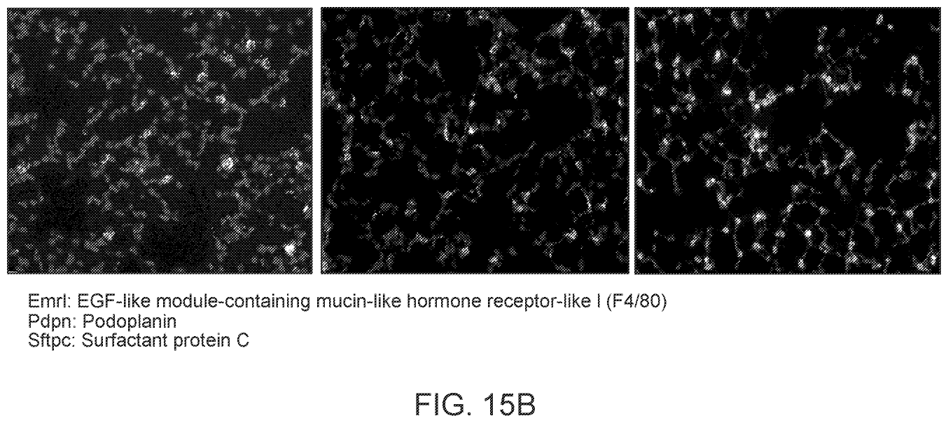

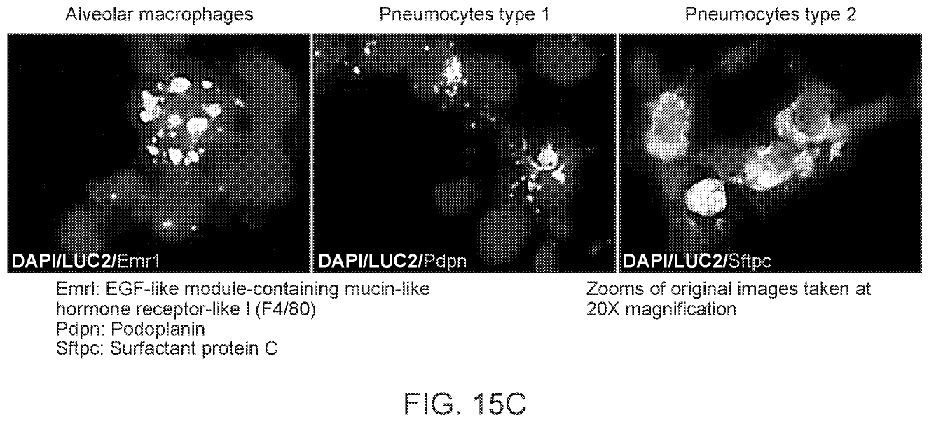

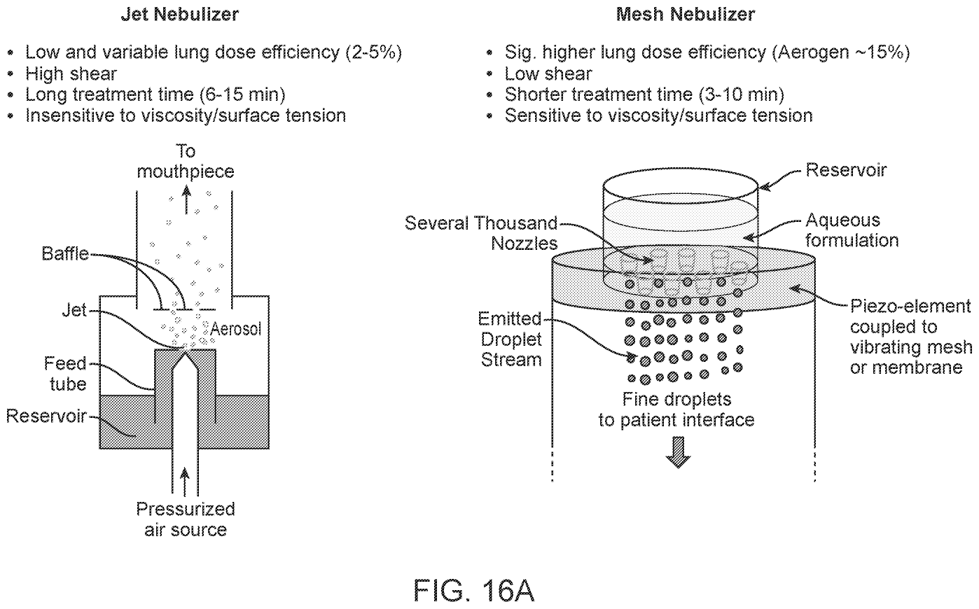

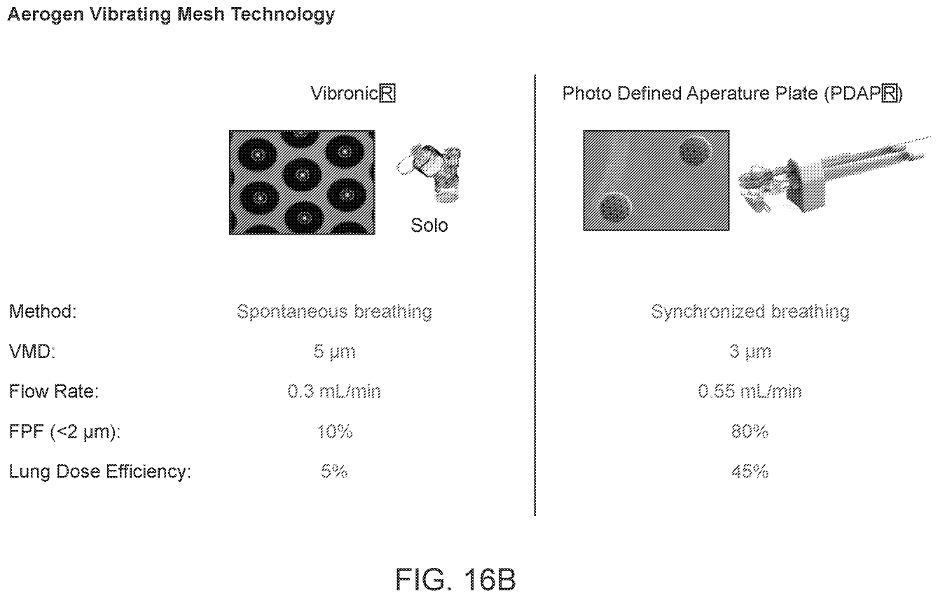

Co-straining of by FISH using BaseScope (ACD) using a combination of Luc2 probes with either EGF-like module-containing mucin-like hormone receptor-like 1 (EMR1), Podoplanin (Pdpn) or Surfactant protein C (Sfptc) was performed to examine co-localization of nanoparticles/mRNA with alveolar macrophage, type I pneumocytes and type II pneumocytes, respectively. Luc2 colocalized with both Pdpn and Sftpc cell markers (FIGS. 15B and 15C).

### Example 10

Product Component Recovery and Activity Post-Nebulization

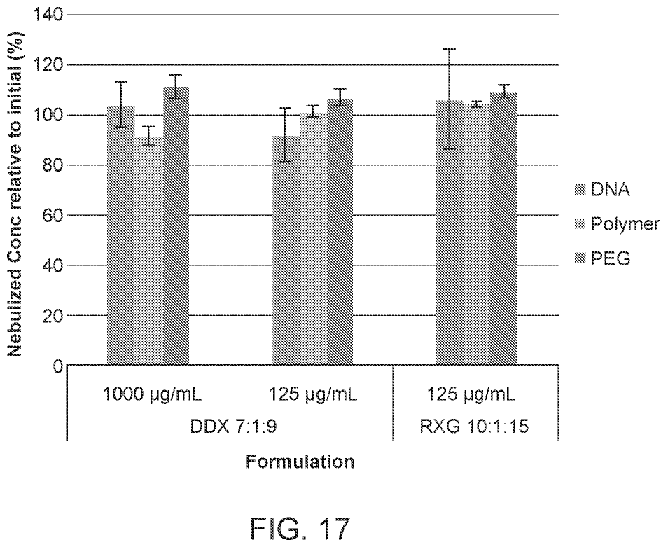

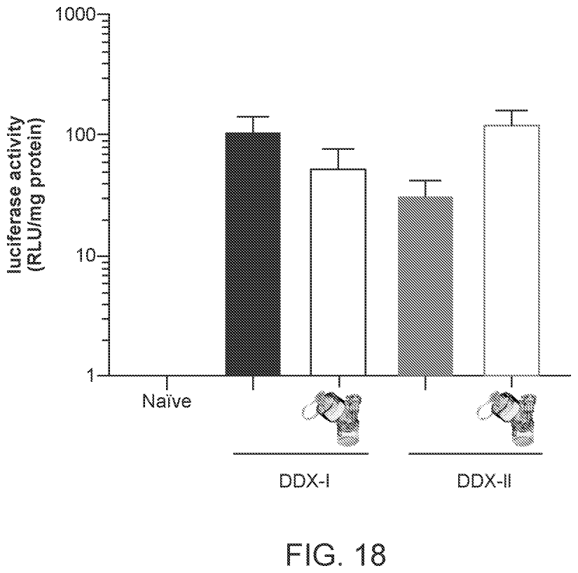

Recovery of two DDX/RXG formulations following nebulization using a vibrating mesh nebulizer were assessed. As seen in FIG. 17, 90-100% of product components (DNA, polymer and PEG) was recovered regardless of the concentration of nanoparticles or type of nanoparticle formulation used. Luciferase activity 24 hours after intranasal delivery using a vibrating mesh nebulizer was assessed. As seen in FIG. 18, luciferase activity in lungs 24 hours after intranasal delivery of polyplex formulations showed nebulization of suspension formulations did not impact the integrity or potency of the suspension formulations.

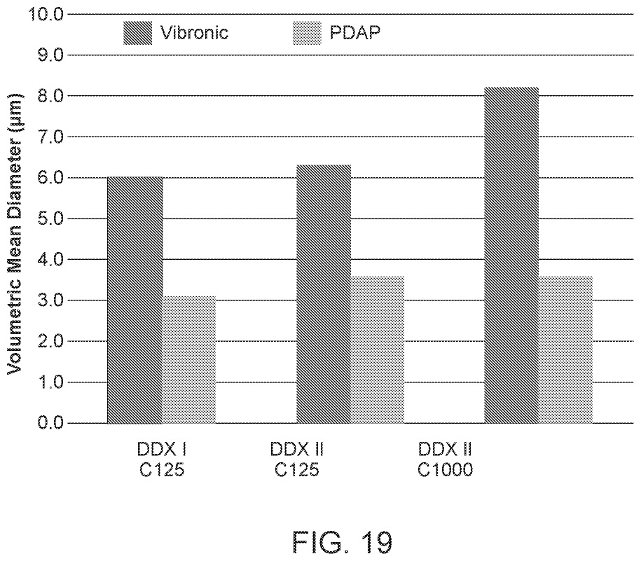

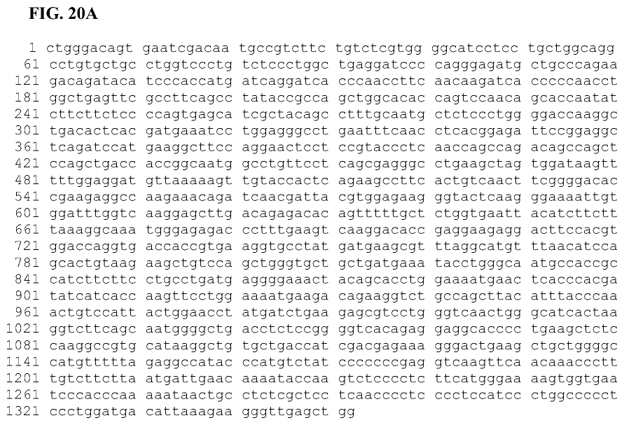

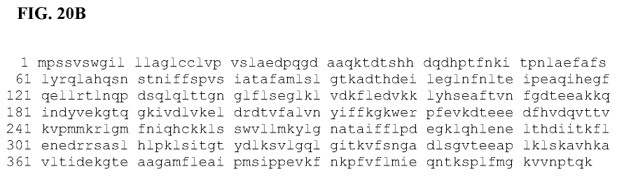

Nebulized DDX polyplex suspensions were characterized as shown in FIG. 19, and in Table 5. PDAP generated uniformly smaller droplets (3-4 μM) compared to Vibronic (6-8 μM). Droplet size remained consistent across all formulations tested.

TABLE 5

| Fine Particle Fraction | Vibronic | PDAP |
|---|---|---|
| FPF < 5 μm | 26-45% | 72-91% |
| FPF < 2 μm | 3-10% | 9-12% |

### Example 11

α-1-Antitrypsin (AAT) in a COPD Mouse Model

CD1 mice transgenic for AAT are exposed to whole cigarette smoke 5 days per week. Control mice are exposed to sham smoke.

### Prolastin Purification

### Polyplex Administration

DDX and/or RXG polyplexes capable of expressing hAAT are administered as described in Example 9 above 24 hours prior to initial smoke exposure and then every 48 hours. The following groups of mice are used: control, control +polyplexn, smoke, smoke+polyplex.

### Collection of Lung Tissue and Lavage Fluid

Animals are sacrificed and the lungs are removed from the chest cavity and lavaged with saline for cell counts, or with water for HPLC analysis of matrix breakdown products. At the 6 month time point, lungs of saline-lavaged animals are inflated with formalin at pressure of 25 cm of water for 24 hours.

### Desmosine and Hydroxyproline Analysis

Analysis for levels of desmosine, a marker of elastin breakdown, and hydroxyproline, a marker of collagen breakdown is assessed by HPLC using established techniques (Li et al., Am J Respiratory and Critical Care Medicine, (1996) 153:644-649.

### Serum A1AT Analysis

Blood is obtained at the time of sacrifice and serum electrophoresis is run using a clinical protein electrophoresis apparatus. hAAT levels in each band are determined using densitometry. Western blots are run to confirm hAAT protein identity.

### Evaluation of Airspace Size

For morphometric analysis, formalin-fixed lungs are serially sectioned in a sagittal plane, and a slice, selected at random, is embedded in paraffin, sectioned, and stained by hematoxylin and eosin. Airspace size can be measured using a standard morphometric grid, with selection of 10-50 random sites counted for each section, and mean linear intercept determined from the summed values of all sites.

### Plasma TNF-α Levels

Plasma TNF-a levels can be determined using a L929 cell assay (Wright et al., Am J Respiratory and Critical Care Medicine, (2002) 166:954-960.

### Animal Studies

4 groups (control, control +polyplex, smoke, smoke+polyplex) are assessed for the number PMN obtained via lavage, lavage hydroxyproline, lavage desmosine at 2, 4, 7, and 30 days.

In some studies, additional controls of albumin in place of polyplex, and hAAT oxidized using established techniques (Churg et al, Lab. Invest., (2001), 81:1119-1131, are used to confirm results of active hAAT.

The 4 groups can also be assessed after extended periods of treatment/smoke exposure as described above. After 6 months exposure, PMN, macrophage levels can be assessed from lavalged samples. Airspace size and TNFa levels can be assessed and compared to controls.

### Example 12

hCFTR Functionality Following Transfection in HEK293 Cells

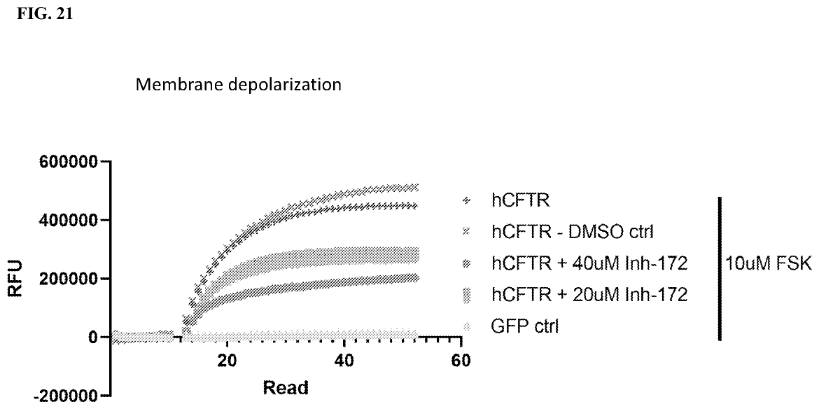

HEK293 cells were seeded into 96-well plates (18,000 cells/well) 24 hours prior to transfection with the indicated plasmids (NTC9385R-hCFTR or NTC9385R-GFP). Transfection was performed using Lipofectamine2000 (ThermoFisher) using 20 ng of plasmid DNA. At 48 hours post-transfection and prior to adding the membrane potential dye, cells were incubated with either DMSO (control), 20 uM or 40 uM of CFTR(Inh)172 (MedChem) or left untreated. Then, the membrane molecular dye (Molecular Probes) was added to the cells for 30 minutes followed with a baseline reading on Envision plate reader (Perkin-Elmer). Cells were stimulated with 10 uM Forskolin (FSK) and membrane depolarization was read on the Envision plate reader (Perkin-Elmer). Results are shown in FIG. 21, where RFU is a measure of the relative fluorescence unit.

### Equivalents

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in the application, in applications claiming priority from this application, or in related applications. Such claims, whether directed to a different invention or the same invention, and whether broader, narrower, equal or different in scope in comparison to the original claims are also regarded as included within the subject matter of the inventions of the present disclosure.

1. A composition comprising a derivatized chitosan nucleic acid polyplex comprising amino-functionalized chitosan and at least one therapeutic nucleic acid for the treatment of a lung disorder, preferably wherein said therapeutic nucleic acid encodes a protein selected from the group consisting of cystic fibrosis transmembrane conductance regulator (CFTR) or a functional fragment thereof and human alphal-antitrypsin protein or a functional fragment thereof.

2. The composition of claim 1, wherein said derivatized chitosan nucleic acid polyplex further comprises a reversible coating comprising one or more polyanion containing block co-polymers.

3. The composition according to claim 1, wherein said amino-functionalized chitosan further comprises a hydrophilic polyol.

4. The composition according to claim 1, wherein said amino-functionalized chitosan comprises arginine.

5. The composition according to claim 2, wherein said hydrophilic polyol is glucose or gluconic acid.

6. The composition according to claim 1, wherein each polyanion-containing block co-polymer comprises at least one polyanionic anchor region and at least one hydrophilic tail region.

7. The composition according to claim 5, wherein each polyanion-containing block co-polymer is an, optionally linear, diblock and/or triblock co-polymer.

8. The composition according to claim 1, wherein said therapeutic nucleic acid is contained within an expression vector.

9. The composition according to claim 7, wherein said expression vector comprises one or more of the following elements:
a. CMV-IE based promoter/enhancer, or a lung cell-specific promotor such as CC10, SP-B, or SP-C,
b. a synthetic Beta-globin-based intron,
c. HTLV-1R
d. kanamycin selection or sucrose-based selection element,
e. an origin of replication.

10. The composition according to claim 7 or 8, wherein said expression vector is comprised within a plasmid selected from the group consisting of: gWIZ, pVAX, NTC8382 NTC8685, or NTC9385R.

11. The composition of claim 1, wherein the therapeutic nucleic acid comprises SEQ ID NO:1 or NM_000492.4, or a functional fragment thereof.

12. The composition according to claim 1, wherein the therapeutic nucleic acid encodes a functional protein comprising SEQ ID NO:2 or NP_000483.3 or a functional fragment thereof.

13. The composition of claim 1, wherein the therapeutic nucleic acid comprises SEQ ID NO:3 or K01396.1, or a functional fragment thereof.

14. The composition according to claim 1, wherein the therapeutic nucleic acid encodes a functional protein comprising SEQ ID NO:4 or AAB59375.1 or a functional fragment thereof.

15. A pharmaceutical composition comprising the composition according to any one of claims 1-14, preferably wherein said pharmaceutical composition is aerosolized.

16. A method for the localized expression of functional protein of interest in an airway epithelial tissue in a patient having a lung disorder, comprising administering to said patient a therapeutically-effective amount of a pharmaceutical composition according to claim 15.

17. A method of treating cystic fibrosis in a patient in need thereof, comprising administering to said patient a therapeutically effective amount of a pharmaceutical composition according claim 15, preferably wherein said patient has a Class 1 CFTR mutation.

18. The method of claim 16 or 17, wherein said pharmaceutical composition is aerosolized, preferably wherein said pharmaceutical composition is delivered using a mesh nebulizer.

19. A liquid formulation comprising derivatized chitosan nucleic acid polyplexes comprising amino-functionalized chitosan and at least one therapeutic nucleic acid for the treatment of a lung disorder in a trehalose solution for delivery as an aerosol via inhalation, preferably using a mesh nebulizer, wherein said polyplexes further comprise a reversible coating comprising one or more polyanion containing block co-polymers.

20. The liquid formulation according to claim 19, wherein said amino-functionalized chitosan further comprises a hydrophilic polyol.

21. The liquid formulation according to claim 19, wherein said amino-functionalized chitosan comprises arginine.

22. The liquid formulation according to claim 20, wherein said hydrophilic polyol is glucose or gluconic acid.

23. The liquid formulation according to claim 19, wherein each polyanion-containing block co-polymer comprises at least one polyanionic anchor region and at least one hydrophilic tail region.

24. The liquid formulation according to claim 23, wherein each polyanion-containing block co-polymer is an, optionally linear, diblock and/or triblock co-polymer.

25. The liquid formulation of claim 19, wherein the polyplexes have an amino to phosphorus (N:P) ratio of between 5:1 and 15:1, or a N:P ratio of 7:1 or 10:1.

26. The liquid formulation according to claim 19 or 20, wherein the amino to anion (N:A) molar ratio is from about 1:3 to about 1.7, more about 1:5, yet more preferably wherein the N:P:A ratio is about 10:1:7; about 10:1:3; or about 10:1:5.

27. The liquid formulation of claim 19, having a DNA concentration of 0.1-2 mg/mL DNA.

28. The liquid formulation of claim 19, wherein the trehalose concentration is between 4% and 6%, preferably about 5%.

29. The liquid formulation of claim 19, having a pH between 5.0 and 8.0.

30. The liquid formulation of claim 19, having an osmolality between 150 and 250 mOsm/kg.

31. The liquid formulation of claim 19, which is free of unbound DNA.

32. The liquid formulation of claim 19, comprising between 75-100% supercoiled DNA, or between 85-95% supercoiled DNA.

33. The liquid formulation of claim 19, having a polydispersity index (PDI) of less than 0.5, 0.4, 0.3 or 0.25.

34. The method of claim 16 or 17, wherein said therapy further comprises treatment with an anti-inflammatory agent, bronchodilator, or antimicrobial agent.


SEQUENCE LISTING (4 sequences)

SEQ ID NO: 1 - DNA, 6070 bp, Homo sapiens
gtagtaggtctttggcattaggagcttgagcccagacggccctagcagggaccccagcgcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatataacaatgctgtattttaaaagaatgattatgaattacatttgtataaaataatttttatatttgaaatattgactttttatggcactagtatttctatgaaatattatgttaaaactgggacaggggagaacctagggtgatattaaccaggggccatgaatcaccttttggtctggagggaagccttggggctgatgcagttgttgcccacagctgtatgattcccagccagcacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgtgaaa

SEQ ID NO: 2 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 3 - DNA, 1352 bp, Homo sapiens
ctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctgg

SEQ ID NO: 4 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK